(19) United States
(12) Patent Application Publication
(10) Pub. No.: US 2024/0324907 A1
(43) Pub. Date: Oct. 3, 2024

(54) SYSTEMS AND METHODS FOR EVALUATING GAIT

(71) Applicant: University of Ottawa, Ottawa (CA)

(72) Inventors: Ryan Graham, Ottawa (CA); Matthew Mavor, Ottawa (CA); Victor Chan, Ottawa (CA); Alexandre Mir-Orefice, Ottawa (CA)

(21) Appl. No.: 18/619,845

(22) Filed: Mar. 28, 2024

Related U.S. Application Data
(63) Continuation-in-part of application No. PCT/CA2023/050446, filed on Mar. 31, 2023.

(51) Int. Cl. A61B 5/11; A61B 5/00; G16H 20/70; G16H 50/20

(52) U.S. Cl. CPC A61B 5/112; A61B 5/0022; A61B 5/4848; A61B 5/6807; A61B 5/7435; G16H 20/70; G16H 50/20; A61B 2505/09; A61B 2562/0219

(57) ABSTRACT

Disclosed herein are systems and methods for monitoring and evaluating a user's gait, comprising receiving, by a computing device having a memory and a processor, from a pair of smart insoles communicatively coupled to the computing device and worn by the user, data measured by one or more types of sensors. The processor segmenting the data into gait-related segmented data comprising one or more of: gait cycle segmentation or activity type, and processing the gait-related segmented data to determine one or more of: gait patterns associated with the segmented data; gait parameters associated with the segmented data; gait phenotypes associated with the segmented data. The device determining, via one or more algorithms a composite movement quality score, based on a combination and interaction of at least two of: the segmented data, the gait patterns, the gait parameters, and the gait phenotypes.

### CROSS-REFERENCE TO RELATED APPLICATIONS

This application claims the benefit of priority to and is a continuation-in-part of PCT Application PCT/CA2023/050446, filed Mar. 31, 2023, entitled “A SYSTEM AND METHOD OF MONITORING GAIT.”

### FIELD OF THE DISCLOSURE

The present disclosure pertains to systems and methods of monitoring and evaluating gait.

### BACKGROUND

The majority of people with multiple sclerosis (PwMS) may experience a walking impairment, which can lead to falls, and have collectively expressed that mobility is a significant contributor to their quality of life (Bethoux and Bennett, 2011; Heesen et al., 2008).

Clinicians employ various tests to assess a PwMS's gait quality, based on clinical outcome parameters measured by the tests that include, but are not limited to, ambulation fatigability, ambulatory index, and the like, which can aggregate to assess a patient in terms of scores such as the expanded disability status scale (EDSS) scores.

Amongst a battery of neurological tests, clinicians may employ clinical walking tests (e.g., the 500-metre walk test, the six-/two-minute walk test, and the timed 25-foot walk and questionnaires (e.g., MS walking scale 12 (MSWS12)) to longitudinally track the progression of MS and evaluate the effectiveness of interventions, including, but not limited to: exercise, assistive device, surgery, pharmacological (Kurtzke, 1983; Phan-Ba et al., 2012; Chen et al., 2021; Decavel et al., 2019; Bethoux et al., 2016; Hobart et al., 2003).

These walking tests are quick to administer and require minimal equipment while providing meaningful outcome measures and generally valid results (Learmonth et al., 2013; Motl et al., 2017; Stellmann et al., 2015).

However, these tests can be burdensome on a patient, as they must be done in-person, causing the patient to travel to a clinician's (or clinicians') offices. The act of visually observing the participant inherently makes some of these metrics subjective; although objective measurements are recorded (e.g., travel time, distance), there may be more objective information that could be collected and used to inform the clinician on their patient's ambulatory status and thus disease progression/regression.

Moreover, these walking tests are only designed to evaluate gross walking ability (i.e., maximum velocity, distance travelled, travel time, perception), not to analyze the many kinematic, kinetic, and spatiotemporal metrics that characterize the human gait pattern.

Existing methods of objective tests for monitoring and assessing gait in a lab, requiring motion cameras and complex equipment, which may not be easily translated to a mobile or portable method which can be run reliably and continuously, without human intervention.

Understandably, most clinicians may not be able to access the prohibitively expensive and sophisticated equipment found in motion capture laboratories, which may be able to capture the above missing data.

Without this access, clinicians cannot evaluate important gait metrics that have been shown to characterize disease progression and significantly differentiate PwMS from healthy participants: velocity, step length, step time, cadence, double support time, swing time, stance time, step width, and gait variability (Givon et al., 2009; Preiningerova et al., 2015; Severini et al., 2017; Shah et al., 2020, Socie et al., 2013; Chitnis et al., 2019; Filli et al., 2018; Monaghan et al., 2021; Sehle et al., 2011).

However, with the emergence of wearable technology, spatiotemporal gait evaluation, once bound to laboratory settings, is becoming increasingly more accessible to clinicians to objectively evaluate their patients' gait patterns during clinical walking tests (e.g. Cheng et al., 2021; Shema-Shiratzky et al., 2019) and daily life (e.g., Block et al., 2017; Chitnis et al., 2019).

Wearable devices are body-worn equipment typically in the form of an inertial measurement unit (IMU; accelerometer, gyroscope, magnetometer (not always present)) and optionally combined with other sensors (e.g., pressure, heart rate, blood oxygen, etc.).

In some cases, pressure sensor data can be leveraged to detect gait events, be used as a substitute for ground reaction forces, measure balance variables (e.g., centre of pressure (COP) area, COP displacement), and be used to measure temporal events (e.g., stance time, double support time, single support time, sway time, cadence).

An accelerometer may allow for the calculation of spatial variables (e.g., step length, step width, step height, distance travelled), and combined with gait events to calculate velocity.

Gyroscope data can be used to calculate turning variables (e.g., mean, max, min turning angle/velocity) at the level of the foot.

By fusing accelerometer, and gyroscope data, (e.g., using an Unscented Kalman Filter or a Madgwick filter), some methods have demonstrated a framework for determining three dimensional angles of the foot, and/or the position and orientation of the foot in space, which can be calculated through all aspects of the gait cycle. From that orientation, distance travelled (i.e., stride length) and foot angle can be understood.

However, the participant must first perform a calibration procedure to orient the sensor to the foot and the fusion algorithm must be robust enough to control for drift over time, possibly by reorienting the sensor during quiet stance.

New and emerging short-range wireless communications technologies such as UWB can be integrated into sensor network systems. In addition to their low cost, low complexity, and lack of interference with other wireless systems, they provide precise self-location information, making this technology useful for tracking. In the context of gait analysis, knowing the location of one sensor relative to another allows for the calculation of foot-to-foot location and variability between gait cycles, step width, step length, and base of support, among others. These variables are important for assessing fall risk as the mediolateral and anteroposterior foot location directly influence a person's base of support, and when combined with force/pressure data, the centre of mass can be derived, which provides useful information for assessing fall risk.

Studies have shown that that spatiotemporal variables are significantly different within the multiple sclerosis (MS) population with respect to EDSS ranges and compared to the healthy populations.

There is less evidence that angle-based metrics are important when assessing PWMS's gait_pattern(s). Additionally, when angle-based, or turning variables, are identified as important, the sensors are located on the trunk.

For PwMS Shah et al. (2020) noted that the most important variables to discriminate from healthy persons are activity variables (i.e., strides per walking bout, strides per hour) and spatiotemporal variables, including gait speed, double support %, swing %, stride duration, cadence, step duration, and stride length (Shah V V., McNames J, Mancini M, Carlson-Kuhta P, Spain R I, Nutt J G, et al. Quantity and quality of gait and turning in people with multiple sclerosis, Parkinson's disease and matched controls during daily living. J Neurol. 2020; 267(4):1188-96.).

Previous studies have also employed wearable devices for tracking basic motion trends in PwMS, demonstrating moderate to strong trends for daily step count, mobility, and sleep compared to clinically derived disability metrics (Block et al., 2019; Sun et al., 2022; Chitnis et al., 2019; Shah et al., 2020; Supratak et al., 2018).

This work is not limited to MS; similar success has been demonstrated in people with Parkinson's disease (e.g., Salis et al., 2023) and cardiovascular disease (e.g., Del Din et al., 2017).

Using wearable devices and other laboratory-grade motion capture technologies, researchers have also been able to implement machine learning (ML) architectures to identify MS disease progression, classify walking phenotypes, identify fall risk, and classify between healthy and PwMS. By frequently capturing a large dataset of PwMS, many of these developed ML architectures can be matured to provide meaningful results and eventually be implemented into clinical practice (Trentzsch et al., 2021; Filli et al., 2018; Meyer et al., 2021; Schumann et al., 2022).

However, a key aspect of any solution is utility. Not all clinicians have a biomechanical or data analysis background that they can rely on to interpret dozens of gait metrics to identify trends of improvement or worsening. Further, patients cannot be actively involved in their healthcare when provided with information they do not understand.

Furthermore, in terms of processing gait data measured by such equipment or by more accessible equipment, there are numerous technical challenges to the processing and identifying trends in the variable mobility data belonging to a person with one or more neurological disorders (the neurological disorders including, but not limited to: Multiple Sclerosis (MS), Parkinson's disease, Alzheimer's disease, Cerebral Palsy (CP), Epilepsy, Motor Neurone Disease (MND), Neurofibromatosis, and the like), or elderly persons, or persons with injuries, walking assisting devices, and the like. These challenges may affect accuracy of computed assessments, and/or the robustness of the algorithms to perform just as well under uncontrolled conditions—“in the wild”.

Diseases affecting the central nervous system (CNS), impairments to the person's mobility are largely dependent on where the scarring is within the CNS, resulting in no single gait description to characterize this population, and resulting in technical challenges in developing algorithms and models that can characterize many different gaits.

In addition, studies have been reporting the measurement of some spatiotemporal variable, such as velocity, step length, step time, cadence, double support time, swing time, stance time, step width, and gait variability, that may contribute to distinguishing between healthy participants and PwMS.

Gait spatiotemporal and balance variables significantly change as PwMS become progressively fatigued, and may be correlated with EDSS scores, and other gait-relevant clinical outcome scoring methods (e.g., Multiple Sclerosis Functional Composite—MSFC, or Artificial Intelligence methods—AI). Other variables, which may include local dynamic stability, sway displacement and distance, stability, turning velocity, turning angle, ground reaction forces, lower limb kinetics, may also be important measurements to obtain when characterizing PwMS's gait patterns.

Data may further be obtained by other variables such as smartwatches or activity trackers, but may not provide the same accuracy and/or may not be conducive to building a robust evaluative framework for persons with abnormal gaits.

Notably, there is no known method of providing frameworks for aggregating and translating at home or in-lab patient motion data to provide clinicians and patients with an easily understandable overview of the data, in the form of a novel composite index (CI) or composite movement quality score, coupled with actionable feedback triggered by the automatic processing and conversion of data to the score.

Therefore, developing a framework which can identify the metrics that are significant, vs. insignificant, and to what extent, and use this information to accurately assess a user's gait, as well as diagnose the assessment in terms of the user's disorder, progression of their disorder, injury, and progression of injury/recovery from injury, are generally not technically feasible with traditional methods of data processing, given the wide spectrum of variables present between patient's having neurological disorders and healthy patients.

Furthermore, once the framework is developed, transforming that framework to an understandable and actionable platform having a useful user interface or graphical user interface (GUI) for both clinicians and patients, is just as variable, and requires significant technical improvements over known methods.

Further, algorithms can be created which may develop a framework for the processing of collected data to provide clinicians with meaningful objective data with minimal human intervention. In this regard, ML can be leveraged to provide composite index (CI) scores from 0-100% to represent a high-level measure of their walking quality and provide data points from which trends can be quickly observed. Similar methods have been successfully implemented in sports research to characterize athletic ability (Ross et al., 2018).

Such technology can provide clinicians with an enhanced ability to intervene (medical or exercise-based) in the progression of the disease to lessen the impact on patients' quality of life. Creating such algorithms that can assess abnormal gaits, or widely varying gaits, is a technical challenge, as there may not be predictable trends in a user's gait (for example, people with MS, people with Parkinson's, and other diseases that may exhibit neurological biomarkers affecting gait, or variability due to people's use of assistive devices such as walkers, canes, and the like). Furthermore, the variability of gait events “in the wild”, or outside of a lab, can be significant roadblocks to providing an accurate assessment of gait.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a system and method for monitoring and evaluating gait.

In accordance with an aspect of the disclosure, there is provided a computer implemented method for evaluating a user's gait, the method comprising: receiving, by a computing device having a memory and a processor, from a pair of smart insoles communicatively coupled to the computing device and worn by the user, data measured by one or more types of sensors; segmenting, by the processor of the computing device, the data into gait-related segmented data comprising one or more of: gait cycle segmentation or activity type; processing the gait-related segmented data, via one or more algorithms stored in the memory of the computing device, to determine one or more of: gait patterns associated with the segmented data; gait parameters associated with the segmented data; gait phenotypes associated with the segmented data; calculating, via one or more algorithms a composite gait quality score, based on a combination and interaction of at least two of: the segmented data, the gait patterns, the gait parameters, and the gait phenotypes.

In some embodiments, the computer-implemented method further comprises: displaying, on a graphical user interface of a user device communicatively coupled to the computing device, the composite gait quality score.

In some embodiments, the graphical user interface comprises one or more of: a clinician portal and a patient smart phone app; wherein the clinician portal and patient app are further configured to receive raw and processed patient data from the system.

In some embodiments, the determining, via one or more algorithms a composite gait quality score, further comprises: training a support vector machine (SVM), of the one or more algorithms, to classify walking data associated with a plurality of participants in one or more groups.

In some embodiments, training the SVM comprises a feature selection step to remove redundant features and identify one or more designated metrics for evaluating gait in a specific patient population.

In some embodiments, the one or more sensor types comprise at least one of: an accelerometer, a gyroscope, a magnetometer, a pressure sensor, and a temperature sensor.

In some embodiments, the parameters comprise gait metrics from said heel strike, foot on floor, heel raise and toe off data.

In some embodiments, the gait patterns comprise a gait signature of the individual from said gait metrics.

In some embodiments, determining the composite gait quality score further comprises: determining a progression over time of the combination and interaction of at least two of: the segmented data, the gait patterns, the gait parameters, and the gait phenotypes.

In some embodiments, determining the composite gait quality score further comprises: an assessment of the user's gait signature and recommendations for improvements.

In some embodiments, determining the composite gait quality score further comprises: an assessment of a change in a patient's neurological disease condition based on measured changes to the patient's gait.

In some embodiments, determining the composite gait quality score further comprises: determining a baseline objective walking quality score of the individual obtained prior to start of a rehabilitation program or immediately following an injury; determining, based on the identified changes in gait pattern, an effectiveness of said rehabilitation program.

In some embodiments, the processing is optimized, by the processor, based on a type of assistive device being used by the user.

In accordance with another aspect, there is provided a system for evaluating a user's gait, the system comprising: a processor; and a memory comprising instructions stored thereon, which when executed by the processor, causes the processor to: receive data measured by one or more types of sensors of a pair of smart insoles worn by the user, the smart insoles in communication with the computing device, segment the data into gait-related segmented data comprising one or more of: gait cycle segmentation or activity type; process the gait-related segmented data, via one or more algorithms stored in the memory of the computing device, to determine one or more of: gait patterns associated with the segmented data; gait parameters associated with the segmented data; gait phenotypes associated with the segmented data; calculate, via one or more algorithms, a composite gait quality score, based on a combination and interaction of at least two of: the segmented data, the gait patterns, the gait parameters, and the gait phenotypes.

In some embodiments, the system further comprises a user device communicatively coupled to the processor, and operable to display on a graphical user interface the composite gait quality score.

In some embodiments, determining, via one or more algorithms a composite gait quality score, further comprises: training a support vector machine (SVM), of the one or more algorithms, to classify walking data associated with a plurality of participants in one or more groups.

In some embodiments, training the SVM comprises a feature selection step to remove redundant features and identify one or more designated metrics for evaluating gait.

In some embodiments, determining is optimized based on a type of assistive device used by the user.

In accordance with another aspect, there is provided a non-transitory computer-readable storage medium comprising instructions stored thereon, which when executed by one or more processors, cause the one or more processors to perform operations for evaluating a user's gait, the operations comprising: receiving data, measured by one or more sensors of a pair of smart insoles worn by the user, the smart insoles in communication with the computing device, segmenting, by a processor of the computing device, the data into gait-related segmented data comprising one or more of: heel strike, foot on floor, heel raise, toe off data, and activity type; processing the gait-related segmented data, via one or more algorithms stored in the memory of the computing device, to determine one or more of: gait patterns associated with the segmented data; gait parameters associated with the segmented data; gait phenotypes associated with the segmented data; calculating, via one or more algorithms a composite gait quality score, based on a combination and interaction of at least two of: the segmented data, the gait patterns, the gait parameters, and the gait phenotypes.

In some embodiments, determining, via one or more algorithms a composite gait quality score, further comprises: training a support vector machine (SVM), of the one or more algorithms, to classify walking data associated with a plurality of participants in one or more groups.

Other aspects, features and/or advantages will become more apparent upon reading of the following non-restrictive description of specific embodiments thereof, given by way of example only with reference to the accompanying drawings.

### BRIEF DESCRIPTION OF THE DRAWINGS

Several embodiments of the present disclosure will be provided, by way of examples only, with reference to the appended drawings, wherein:

FIG. 1 illustrates a block flow diagram of a system and method in accordance with an embodiment of the disclosure;

FIG. 2 provides a block flow diagram of a system and method in accordance with an embodiment of the disclosure;

FIG. 3A provides details with respect to determining a composite gait quality score;

FIG. 3B illustrates how a person's gait quality fits within a population, in accordance with one embodiment;

FIG. 4 illustrates a flow diagram of methods for monitoring gait, in accordance with one embodiment;

FIG. 5 illustrates a flow diagram detailing sensor inputs, outputs, algorithms and clinician portal and patient app, in accordance with one embodiment;

FIG. 6 illustrates a flow diagram illustrating algorithms and progression algorithms, in accordance with one embodiment;

FIG. 7A provides a non-limiting illustrative example of the difference in gait pattern of unassisted vs assisted walking measured with the system of an embodiment of the disclosure;

FIG. 7B illustrates a dysfunctional gait pattern measured with the system of an embodiment of the present disclosure. The gait detection method can segment the data into distinct gait cycles, and each gait cycle into the different phases (heel strike, foot on floor, heel raise, and toe off) based on the measured, raw plantar pressure, accelerometer, and gyroscope data retrieved from the instrumented insoles.

FIG. 8 illustrates a visual representation of the difference in the patterns of the gyroscope pitch between the left (circumduction) and right feet (see area circled);

FIG. 9A illustrates unassisted (left circumduction) gait pattern of an individual whose left leg expresses circumduction (i.e., rotates about the hip to clear their foot as they do not have sufficient dorsi flexion strength) determined using a method in accordance with an embodiment.

FIG. 9B illustrates the difference in patterns of the Yaw gyroscope;

FIG. 10A illustrates ground truth whole-body motion capture, in accordance with an embodiment of the disclosure;

FIG. 10B illustrates assisted gait pattern determined using a method of an embodiment of the present disclosure;

FIG. 11 illustrates graphically balance analysis using a method of an embodiment of the disclosure. Balance metrics can identify changes due to eyes being open and closed (i.e., reducing feedback to the central nervous system to maintain balance). A larger area is indicative of a reduced balance quality;

FIG. 12 illustrates balance analysis with the system of an embodiment of the present disclosure. By extracting and processing the same data as illustrated in FIG. 11, the velocity of the centre of pressure displacement was assessed. Higher values are correlated with an increase in fall risk;

FIG. 13A illustrates the ground truth method used (MVN Link, Xsens, Netherlands) when outside of the motion capture laboratory;

FIG. 13B illustrates the foot position of an instrumented insole. The participant performs a ˜125 metre walk with a stair ascend and descend portion. Foot position is calculated using a Madgwick sensor fusion algorithm;

FIG. 14 illustrates provides an illustrative example of a composite score of gait quality calculated over time of an individual, in accordance with one embodiment;

FIG. 15 depicts the raw sensor data from the instrumented shoe insole and the gait event overlay of a participant performing an ˜125 metre walk with a stair ascend and descend portion, in accordance with one embodiment;

FIG. 16 illustrates depicts a hypothetical machine learning clustering result of principal component scores to identify gait phenotypes (e.g., healthy, ataxic, hemipelagic, etc.), in accordance with one embodiment;

FIG. 17A and FIG. 17B and FIG. 17C illustrate one or more steps set forth by a user interface of a mobile app of an embodiment of the present disclosure;

FIG. 18 illustrates graphically, one or more aspects of a composite movement or gait quality score in accordance with one embodiment of the present disclosure;

FIG. 19 illustrates a box diagram of one or more aspects of a composite movement or gait quality score, in accordance with one embodiment of the present disclosure;

FIG. 20A illustrates an aspect of a user interface in accordance with an embodiment of the disclosure; and

FIG. 20B illustrates an aspect of a user interface in accordance with an embodiment of the disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

The expressions “gait pattern” as used herein and throughout this disclosure, refer to a general high-level explanation of someone's walking movement on a stereotypical/population level. Certain general information may be obtained from the gait pattern of an individual, such as and without limitation: gender, body size, emotion, disability status, etc. However, a gait pattern may not be used to identify the individual.

The expression “gait signature” as used herein and throughout this disclosure, refer to a specific representation of an individual's walking movement that can be used to identify an individual (i.e., can be used as a form of biometric identification). Changes to the gait signature in response to stimuli (e.g., perturbation, speed change) are specific to that individual.

Systems and methods are disclosed herein which may address the technical difficulties in collecting the data as described above with short-range wireless communications coupled with insole sensors, processing the data collected via these methods, and developing frameworks for converting the collected data into gait assessments and actionable insights derived from gait assessments.

Systems and methods disclosed herein may address technical challenges in the development of a framework which may collect gait patterns using instrumented shoe insoles, analyze those data to calculate spatiotemporal gait metrics, and provide an interpretable overarching CI score (0-100%) that clinicians can use to identify trends and responses to interventions, and that patients can use to objectively observe their walking quality trends and advocate for their healthcare.

Systems and methods disclosed herein may address technical challenges in the tools currently available for monitoring and/or evaluating a user's gait, by employing short-range wireless communications coupled with insole sensors, in order to provide users with a platform to evaluate their symptoms at home.

### Systems and Methods for Monitoring Gait (2023)

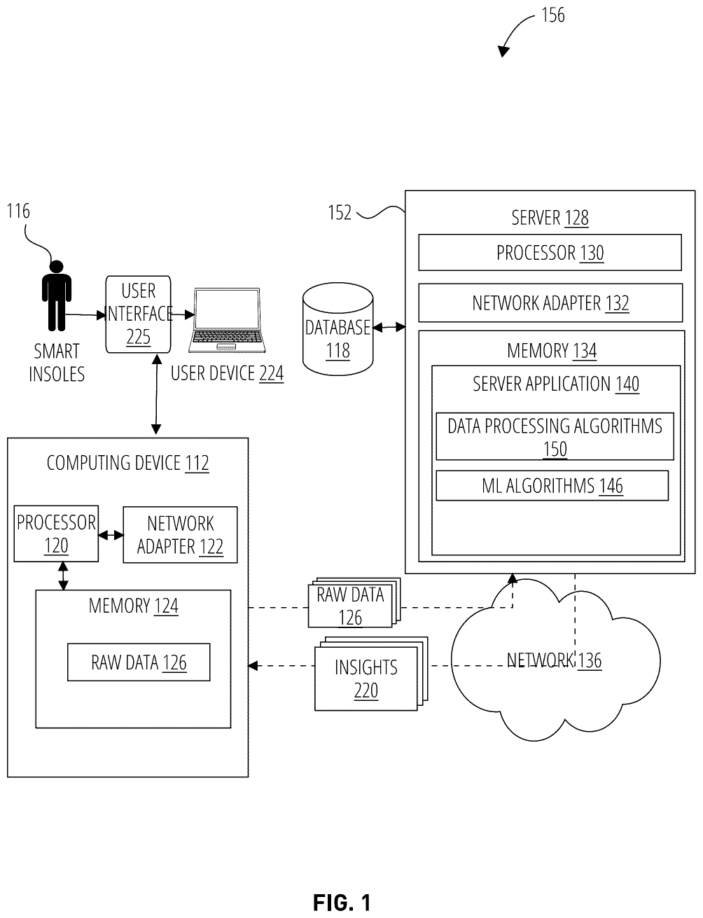

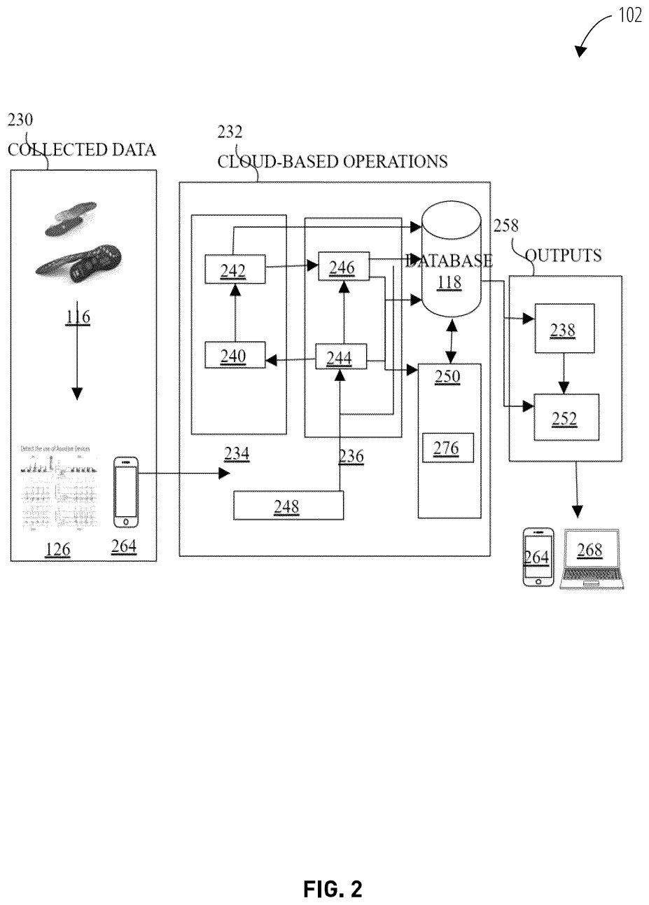

FIG. 1 and FIG. 2 illustrate block flow diagrams of a system 102 and method for analyzing gait and MS progression, in accordance with an embodiment of the disclosure.

An object of the present disclosure is to provide a system and method of monitoring gait.

In accordance with another aspect of the present disclosure, there is provided a system comprising a computing device configured to perform the methods of the present disclosure.

In accordance with another aspect of the present disclosure, there is provided a non-transitory, computer readable memory and/or medium having recorded thereon statements and instructions for execution by a computer the methods of the present disclosure.

As illustrated, a user wearing smart insoles 116 performs walking motions that are recorded as raw data 126 by one or more sensors embedded in the smart insoles 116. In certain embodiments, the smart insoles are in wireless communication, e.g., via Bluetooth, with a user device 224, such as a smart mobile device such as a smart phone, tablet or smart watch comprising a mobile application.

In such embodiments, the smart mobile device receives the raw data 126 collected by the smart insoles and sends the data and optionally other data such as data related to cognitive and dexterity tests to a computing device 112, optionally a cloud-based computing device 152, for analysis with one or more algorithms 146, 150, and optionally storage in a database 118, optionally a cloud-based database 118.

Following analysis, the analyzed data and/or other output may be saved to a database 118, optionally a cloud-based database 118 and/or feedback and insights 220 may be provided to the user and/or other parties such as the user's medical providers. The feedback may be displayed via the mobile application and/or web portal via the user device 224.

Exemplary feedback and insights 220 include but are not limited to alerts or notifications, for example alerts or notifications related to increased fall risk or changes in gait or disease progression, recommendations with respect to treatment programs, rehabilitation programs and/or assistive devices etc.

The raw data 126 collected by the insoles 116 may be received by the computing device in real-time or in batches.

In certain embodiments, the method further comprises one or more steps of collecting the 126 with smart insoles 116. In certain embodiments, the insole data collection is automatic when the insoles are being worn and paired with a smart device comprising the mobile app 1772 of the present disclosure.

In certain embodiments, the insole data collection is in response to user input or at predetermined times. The data collection may be continuous when the insoles are in use or intermittent. In certain embodiments, the method further comprises collecting other data related to cognitive and/or dexterity assessments. For example, cognitive and dexterity tests may be collected through a mobile app 1772 and/or user interface 225 on a user's device 224, by completing surveys, cognitive assessments and upper limb dexterity tests. In certain embodiments, upper limb dexterity tasks and hand to eye coordination is measured using in app 1772 games. In certain embodiments of the system and methods of the present disclosure, a user interface 225 of the user device 224 sends a notification to the user, optionally at predetermined time periods, for example scheduled by a healthcare provider, to complete walking, cognitive and/or dexterity tasks for data collection.

FIG. 2 illustrates in greater detail one or more algorithms that may be employed by the systems and methods of the present disclosure, in order to synthesize a framework for evaluating an individual's gait signature/pattern, and/or in evaluating changes to an individual's gait pattern over time may be used to monitor the progression of a neurological disorder, such as MS, which impacts gait; monitoring for increased risk of falls; and/or the monitoring effectiveness of rehabilitation program/post injury recovery.

In certain embodiments, the raw data 126 collected by the 116 is sent to a cloud-based computing device 232.

Broadly, there are four different kinds of algorithms employed by the systems and methods of the disclosure. Raw data processing algorithms 248 take raw data and segments it. Algorithms for gait pattern classification 236 takes segmented data and classifies it based detected patterns. Algorithms for gait parameters 234 receive classified data and segments it further based on the type of gait, then assess the gate in terms of its performance against key metrics, such as stride, time, stride length, asymmetry, cadence, and the like. Gait pattern classification 236 may also receive raw data or data sorted by the gait parameters 234 algorithm, to phenotype the gait using AI and pattern classification. A database 118 then receives classified data from the gait pattern classification 236 algorithms, and sorts it using a fourth type of algorithm-composite score, which is a novel method of aggregating the outputs to score a user's gait data from a given session in terms of movement quality. The score can be displayed in outputs 258 to a user device in the form of progression graphing (showing the change in gait or gait events over time), and insights 220 or feedback, geared selectively to either a user (patient) or a clinician.

Raw data processing algorithms may comprise: a group 1 algorithm: raw data processing 248), wherein raw data 126 may be segmented, by the computing device, into different activities or tasks of interest. Non-limiting examples of activities include but are not limited to standing, walking, turning, stair ascend/descend, etc. In certain embodiments, the machine learning algorithms employ a sliding window approach. A window refers to a predetermined time period (determine based on the nature of the data; e.g., 1.5 seconds). Then, all data from a window is input to an algorithm, and the activity the data corresponds to is determined (i.e., the output). This window will “slide” forward over the data with a pre-set, constant step size (e.g., 0.5 seconds).

In an embodiment, the machine learning model architecture is comprised of convolutional and long short-term memory (LSTM) layers with a softmax decision layer, selected for their suitability for time series classification problems. Once these decisions are made, the data is segmented, optionally automatically, into the various activities. One purpose of segmenting the data into various activities is to ensure that the appropriate algorithms are utilized for the given activity (i.e., walking, standing, stair navigation, turning, etc.).

For example, if someone is walking, then stops at a streetlight, that standing portion of the data is not analyzed by the walking algorithms, so their step time, cadence, velocity, etc. are not negatively affected. Another purpose will be to identify segments of data suitable for automatic assistive device detection (e.g., walking).

In another embodiment, the machine learning model is a fully connected neural network comprised of dense hidden layers with rectified linear unit (ReLU) activation functions and a softmax decision layer, which may provide a more robust architecture for evaluating large variability in measurements taken in the wild.

In other embodiments, the machine learning (ML) model architecture may comprise a combination of one or more of convolutional, LSTM and dense layers with a decision layer. In another embodiment, the ML model architecture may further comprise a decision-making engine which decides which should be employed in the ML model architecture for activity segmenting.

An advantage of employing one or more hidden layer types (i.e., convolutional, LSTM, dense) is that it increases the flexibility and/or adaptability of the model. For example, if the model receives new data that presents challenges to classification that it has not yet resolved (e.g., a gait phenotype that has abnormalities that manifest over time periods that are significantly longer than a previously monitored window length), the decision-making engine may employ a new requirement to the model architecture, requiring layers with recurrent units like LSTM or similar.

Methods for training a machine learning model to recognize abnormal gait patterns in the wild are disclosed herein.

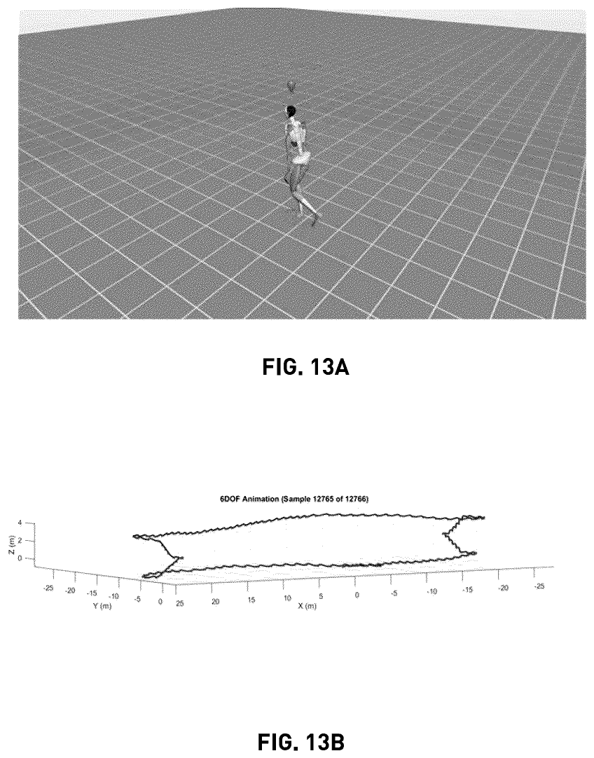

In order to segment data, ground truth labels are needed to train the algorithms 144, 150. Generating ground truth labels requires manually identifying labels using external 3rd party data sources (e.g., video, other motion capture device). An example is illustrated in FIG. 13A.

Given the significance of not only segmenting and removing irrelevant data (i.e. standing at a streetlight while monitoring walking), and segmenting data into different categories, algorithms 144, 150, must be trained to recognize different activities and/or data to be removed.

Identifying these data segments for a person's mobility data measured in the wild is inherently more difficult than in a lab, because the algorithm is not informed by what the person is doing. This challenge may be overcome by the embodiments disclosed herein, by collecting data in a lab under controlled conditions and pseudo random conditions, together with the 3rd party data source to generate the labels needed to train. To ensure robustness of the algorithms 144, 150, many different activities are simulated, and many different environments are used to train the algorithm.

Data from group 1 algorithms 248 may be sent to one or more algorithms for gait pattern classification 236. Group 2 algorithms 244 evaluate whether a user is walking with assistance, and what kind of assistive device they are using. Walking patterns between assisted (e.g., use of a cane, walker or other assistive device) and unassisted walking may be considerably different. Accordingly, in certain embodiments, the method of the present disclosure may automatically determine what kind of assistive device is being used (if any), and on which side of the body (when applicable) prior to detecting gait. Group 5 algorithms 246 may determine one or more gait phenotypes such as healthy, spastic, ataxic, and the like, from either raw, segmented, or processed gait pattern data 236.

In some embodiments, using group 2 algorithms 244, the data related to walking is analyzed, optionally automatically, to determine what kind of assistive device is being used (if any), and on which side of the body (when applicable). In specific embodiments, a machine learning algorithm is used to automatically recognize what kind of assistive device is being used (if any), and on which side of the body (when applicable). In certain embodiments, the method determines if the walking is unassisted, with a walker, with a cane, with an ankle foot orthosis. In embodiments where the assistive device can be unilateral (e.g., a cane and an ankle foot orthosis), the method further determines which side the assistive device is used on. In other embodiments, information related to any assistive devices is inputted or has been previously provided. For example, the information related to any assistive devices may be entered with other personal details when configuring the mobile device app 1772 or the information may be requested/confirmed each time the mobile device app is used.

In certain embodiments, gait parameter algorithms 234 may comprise a group 3 algorithm 240: gait detection algorithm. The group 3 algorithm 240 evaluates each foot as being in one of four phases: heel strike (HES), foot on floor (FOF), heel raise (HER), or toe-off (TOF). These phases are triggered based on a set of pressure threshold values redefined for each walking session, predefined accelerometer and gyroscope thresholds, and are reliant on the preceding events. In certain embodiments, the threshold values used to identify phase of gait is dependent on the assistive device used, if any. In some embodiments, gait detection data 240 is further processed by a group 4 algorithm 242: assessment. The group 4 algorithm determines key gait metrics and evaluates the gait detection data 240 against them. Some key gait metrics may comprise stride time, stride length, cadence, asymmetry, and the like. In certain embodiments, the methods of the present disclosure detect gait from the data related to walking. In specific embodiments, the gait detection algorithm is optimized, optionally automatically, based on assistive device used.

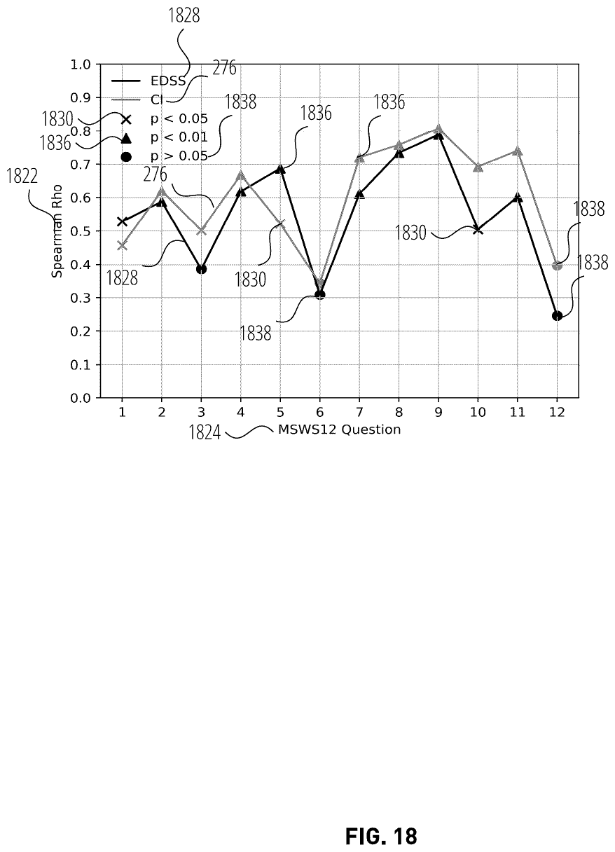

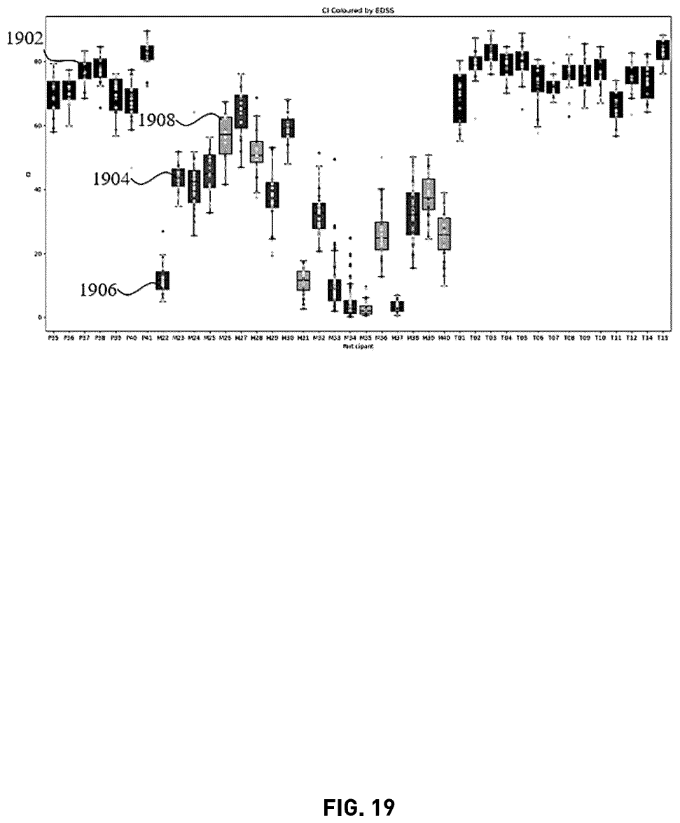

In certain embodiments, the present methods comprise a group 6 algorithm 250 for providing a framework which may calculate a composite movement quality score 276 based on various outputs from the algorithms of groups 1-4, such as outputs of activity classification, assisted walking determination, gait detection, gait assessment and gait phenotyping, and optionally information from previous walking sessions retrieved from the database. In certain embodiments, this score will be a single interpretable number between 0 and 100% and can be used to describe an individual's gait pattern in respect to the population (i.e., z-score). Clinicians may visualize this score based on different populations, such as their patient's mobility (e.g., EDSS score), age, phenotype, assistive device use, etc. This composite movement quality score 276 may be used to track an individual's change from baseline over a period of days, weeks, months, or years. Novel methods of providing a framework for developing a composite index for gait type and health will be described in FIG. 18 and FIG. 19.

In various embodiments, there are disclosed herein computer implemented methods for determining and evaluating an individual's gait signature/pattern, determining and evaluating changes in an individual's gait signature/pattern, and/or determining and evaluating an individual's response to physical therapy or surgery following an injury, based on the individual's gait signature/pattern and/or changes in it. In some embodiments, evaluating the individual's signature gait pattern further comprises assigning the determined signature gait pattern a composite movement quality scores 276 and communicating it to the individual or their clinician via a mobile device or computing device. In some embodiments, assigning the determined signature gait pattern a composite movement quality scores 276 comprises entering the individual's gait data, measured by one or more sensors integrated into, embedded (removably or permanently), or communicatively coupled to, a pair of smart insoles, into one or more algorithms, which may comprise machine learning ml algorithms 144, trained on both healthy users' gaits, and persons with abnormal gaits, such as PwMS.

The methods may comprise one or more steps, including, but not limited to: (a) wirelessly receiving, by a computing device, data, optionally automatically, collected by a pair of smart insoles worn by the individual in wireless communication with the computing device, the smart insoles comprising one or more sensors selected from the group consisting of accelerometer, gyroscope, magnetometer, pressure sensors and ultra-wide band; (b) segmenting, by the computing device, optionally automatically, data related to gait into heel strike, foot on floor, heel raise and toe off data; (c) determining, by the computing device, optionally automatically, gait metrics from said heel strike, foot on floor, heel raise and toe off data; and (d) determining, by the computing device, optionally automatically, the gait signature of the individual from said gait metrics.

Additionally, sensors may comprise temperature sensors which may alert a user as to the sensors' warm up status.

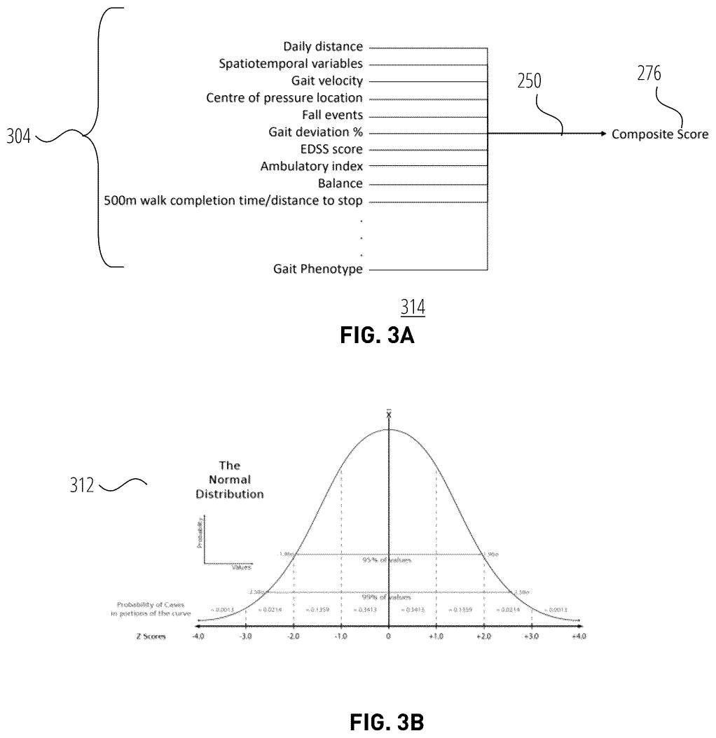

FIG. 3A illustrates one or more variables, which may be processed by a group 6 algorithm: composite score 250, to provide an output comprising one or more composite movement quality scores 276.

A composite score 276 may be calculated using one or more variables 304, both changing and static. The variables 304 may include, but are not limited to, a user's: daily distance, spatiotemporal variables, gait velocity, centre of pressure location, fall events, gait deviation %, EDSS score, ambulatory index, balance, 500 m walk completion time/distance to stop, gait phenotype, and the like. The variables 304 may be automatically or manually retrieved/entered by from raw data 126, from the algorithms 1-4, a patient's medical records, or inputs from the patient and/or clinician.

In certain embodiments, to calculate the composite movement quality score 276, a weighted average of gait metrics (evaluated by gait assessment algorithm group 4 242), phenotypes, and assistive device use, as well as a series of linear regressions and scores from previous sessions, are used to fit the individual within the population of interest. In certain embodiments, individuals will be automatically compared to all populations of interest and logged into the database.

It can be seen in FIG. 3B that, in some embodiments, a user's composite score 276 may be compared with a plurality of other users' composite scores, in order to evaluate it. Comparison may be performed by plotting the user's composite score against the normal distribution 312 of a plurality of users' composite scores and noting its deviation from the mean. Other methods, known in the art, of evaluating a difference between a user's score 276 and an aggregation of other users' scores may be employed instead.

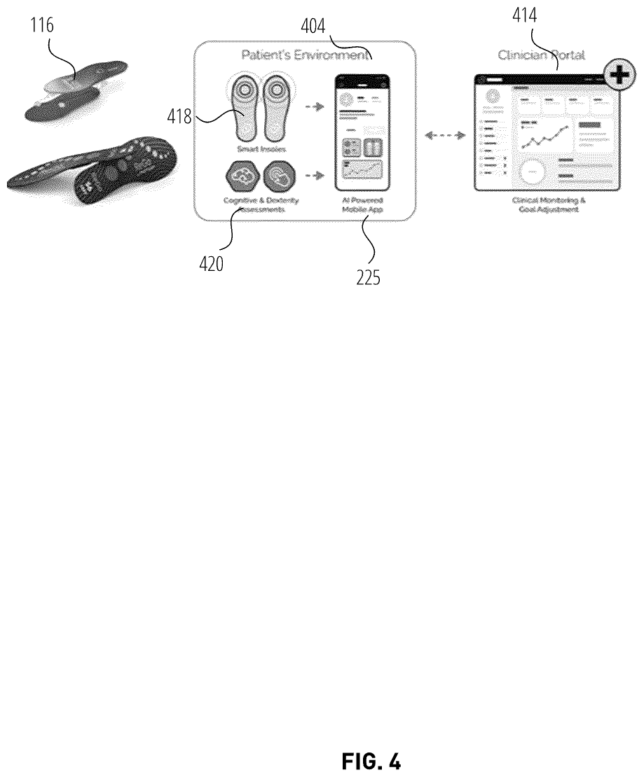

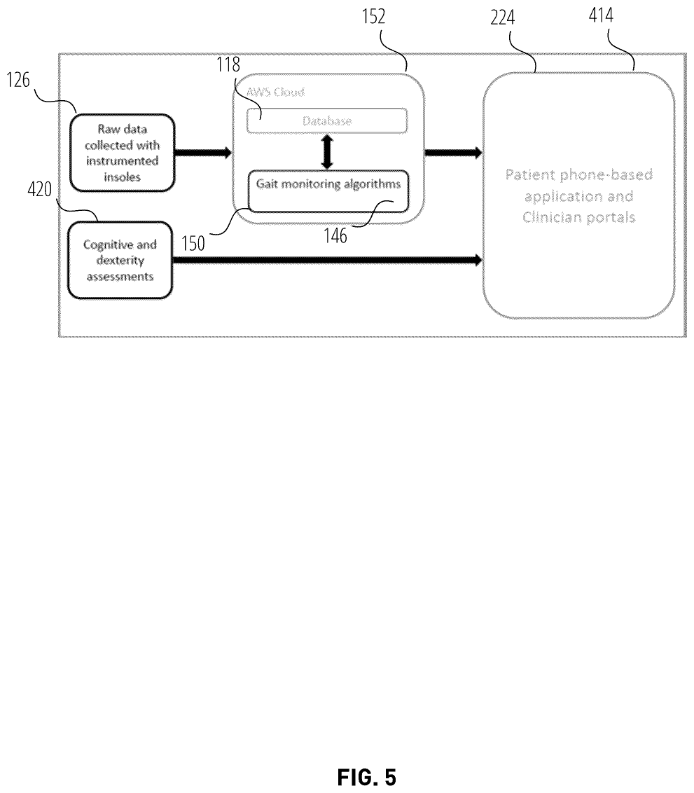

FIG. 4 and FIG. 5 illustrate, schematically, an exemplary flow diagram detailing sensor inputs from smart insoles 116, outputs, algorithms and clinician portal and patient app, in accordance with one embodiment.

In certain embodiments, a patient environment 404 may comprise a user interface 225, coupled to the cloud-based computing device 152 as described in FIG. 1 and FIG. 2. The cloud based computing device receives a patient's gait-related raw data 126 from sensors in the insoles 116, and/or cognitive and dexterity data from cognitive and dexterity assessments 420 (performed in the user interface 225 or received from an external source), and processes the data using the one or more algorithms 146, 150, as described in FIG. 1 and FIG. 2. The processed data is sent to a clinician portal 414, where a clinician may view real time and/or trending assessments of the patient's gait.

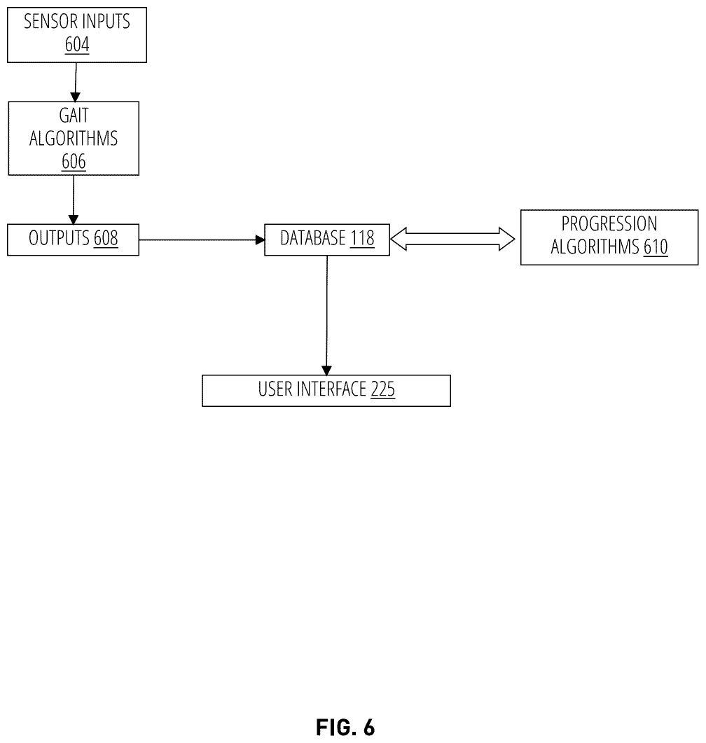

FIG. 6 illustrates systems and methods for monitoring an individual's gait signature/pattern,

Below are exemplary computer implemented methods according to one or more embodiments of the present disclosure.

Generally, sensor inputs 604 are received by a computing device 112 and processed by one or more gait algorithms 606, which may comprise any one of the algorithms described in FIG. 1 and FIG. 2, and sent to a database 118 as outputs 258. The outputs may be further processed by one or more progression algorithms 610, which may comprise any one of the algorithms described in FIG. 1 and FIG. 2, modified to further process the outputs 258 to determine any changes in the user's gait over time or with respect to other users' gaits. The progression data is converted to one or more composite movement quality scores 276, and sent to a user interface 225, such as a patient environment (accessible online or via a smart phone, for example) or a clinician portal (accessible online or via a smart phone, for example).

In accordance with an aspect of the present disclosure, there is provided a computer implemented method for determining an individual's gait signature/pattern; the method comprising: (a) wirelessly receiving, by a computing device, data, optionally automatically, collected by a pair of smart insoles worn by the individual in wireless communication with the computing device, the smart insoles comprising one or more sensors selected from the group consisting of accelerometer, gyroscope, magnetometer, pressure sensors and ultra-wide band; (b) segmenting, by the computing device, optionally automatically, data related to gait into heel strike, foot on floor, heel raise and toe off data; (c) determining, by the computing device, optionally automatically, gait metrics from said heel strike, foot on floor, heel raise and toe off data; and (d) determining, by the computing device, optionally automatically, the gait signature of the individual from said gait metrics. In certain embodiments, the gait phenotype of the individual is determined. In some embodiments, the method is a real-time method. Exemplary gait phenotypes include but are not limited to healthy, ataxic, spastic, hemiplegic using the gait measures, and the direct signals with phases.

In another aspect of the present disclosure, there is provided a computer implemented method of monitoring changes in gait pattern of an individual; the method comprising: (a) wirelessly receiving, by a computing device, data, optionally automatically, collected by a pair of smart insoles worn by the individual in wireless communication with the computing device, the smart insoles comprising one or more sensors selected from the group consisting of accelerometer, gyroscope, magnetometer, pressure sensors and ultra-wide band; (b) segmenting, by the computing device, optionally automatically, data related to gait into heel strike, foot on floor, heel raise and toe off data; (c) determining, by the computing device, optionally automatically, gait metrics from said heel strike, foot on floor, heel raise and toe off data, optionally the gait metrics are selected from stride time, stride length, cadence and asymmetry; (d) determining, by the computing device, optionally automatically, the gait signature of the individual from said gait metrics; and (e) comparing, by the computing device, optionally automatically, the gait signature of the individual to a baseline gait signature of the individual to identify changes in gait pattern. In certain embodiments of the above method, the method further comprises (f) sending, optionally automatically, a notification to the individual and/or to the individual's healthcare provider regarding any changes in the gait pattern, optionally the notification includes recommended interventions. The interventions may include a change in treatment/rehabilitation plan, change in use of assistive device(s). In certain embodiments, the method includes implementation of the recommended interventions.

In accordance with another aspect of the present disclosure, there is provided a computer implemented method of monitoring (i) progression of a neurological disorder which impacts gait/(ii) for increased risk of falls; the method comprising: (a) wirelessly receiving, by a computing device, data, optionally automatically, collected by a pair of smart insoles worn by the individual in wireless communication with the computing device, the smart insoles comprising one or more sensors selected from the group consisting of accelerometer, gyroscope, magnetometer, pressure sensors and ultra-wide band; (b) segmenting, by the computing device, optionally automatically, data related to gait into heel strike, foot on floor, heel raise and toe off data; (c) determining, by the computing device, optionally automatically, gait metrics from said heel strike, foot on floor, heel raise and toe off data, optionally the gait metrics are selected from stride time, stride length, cadence and asymmetry; (d) determining, by the computing device, optionally automatically, the gait signature of the individual from said gait metrics; and (e) comparing, by the computing device, optionally automatically, the gait signature of the individual to a baseline gait signature of the individual to identify changes in gait pattern, wherein an increase in frequency of abnormal stride patterns is indicative of progression of said neurological disorder/increased risk of falls. In certain embodiments, the above method comprises (f) sending, optionally automatically, a notification of the individual and/or the individual's healthcare provider if there is progression of the neurological disorder and/or increased risk of falls, optionally the notification includes recommended interventions. The interventions may include a change in treatment/rehabilitation plan, change in use of assistive device(s) and/or fall prevention strategy. In certain embodiments, the method includes implementation of the recommended interventions.

In accordance with another aspect of the present disclosure there is provided a computer implemented method of monitoring effectiveness of rehabilitation program/post injury recovery; the method comprising: (a) wirelessly receiving, by a computing device, data, optionally automatically, collected by a pair of smart insoles worn by an individual in a rehabilitation program, in wireless communication with the computing device, the smart insoles comprising one or more sensors selected from the group consisting of accelerometer, gyroscope, magnetometer, pressure sensors and ultra-wide band; (b) segmenting, by the computing device, optionally automatically, data related to gait into heel strike, foot on floor, heel raise and toe off data; (c) determining, by the computing device, optionally automatically, gait metrics from said heel strike, foot on floor, heel raise and toe off data, optionally the gait metrics are selected from stride time, stride length, cadence and asymmetry; (d) determining, by the computing device, optionally automatically, the gait signature of the individual from said gait metrics; and (e) comparing, by the computing device, optionally automatically, the gait signature of the individual to a baseline gait signature of the individual obtained prior to start of the rehabilitation program or immediately following the injury to identify changes in gait pattern, wherein a decrease in frequency of abnormal stride patterns is indicative of effectiveness of said rehabilitation program or injury recovery. In certain embodiments, the above method further comprises (f) sending, optionally automatically, a notification of the individual and/or the individual's healthcare provider regarding effectiveness of said rehabilitation program or status of the injury recovery, optionally the notification includes recommended changes to rehabilitation program. The interventions may include a change in treatment/rehabilitation plan. In certain embodiments, the method includes implementation of the recommended interventions.

In the case that the one or more gait algorithms 606 cannot solve for a gait cycle, the phase will be repeated even when not appropriate and a dysfunctional label is put in its place so those cycles can be removed in subsequent algorithms.

Preferably, the gait algorithms 606 of the present disclosure comprise technical improvements which mitigate the algorithm's 606 inability to solve for one or more gait labels. Presently, a decision-making engine of the algorithm 606 finds all toe off events within the data being processed, then works within each gait cycle (toe off to toe off) so that any errors are localized within a single gait cycle, rather than cycling through the same on repeat, until it can solve for a gait event again. This improvement results in a faster, more efficient, and more robust system.

In certain embodiments of the above methods, the phases of a gait cycle comprise heel strike, foot on floor, heel raise and toe off; and step (b) comprises comparing said data to pre-determined thresholds for each phase of a gait cycle; and segmenting the data into data related to gait into heel strike, foot on floor, heel raise and toe off data based on the comparison.

In certain embodiments of the above methods, the gait metrics are temporal metrics, optionally calculated using the indices of the gait phases from both feet in the time domain.

In certain embodiments of the above methods of the disclosure, the gait metrics are spatial metrics, optionally calculated by using the indices of the gait phases from both feet to apply sensor fusion (i.e., Madgwick filter) to the accelerometer and gyroscope data, optional ultra-wideband, and deep learning approaches such as neural networks (long-short term memory, convolutional, etc.).

In certain embodiments of the above methods of the disclosure, the gait metrics are spatiotemporal (time and distance-based, velocity), optionally calculated by taking the integral of the accelerometer signals over time between gait phase indices and by multiplying temporal and spatial metrics.

The gait detection algorithm 606 assesses the pressure, gyroscope, and accelerometer values based on the maximum values observed within this specific batch of data. In certain embodiments, for consistency, the data presented to the gait detection algorithm will begin with the right foot in the FOF phase and the left foot in TOF (i.e., right foot single support). In certain embodiments, the assessment is as follows:

The foot is considered to be in the FOF phase when the values from the heel, toe, or midfoot pressure sensors are greater than or equal to their threshold, the values of the vertical accelerometer and pitch gyroscope are within their respective bounds (±0.8 G and ±10 degrees/second, respectively), and the previous phase is HES or FOF.

The foot is considered to be in HER when the pressure value from the heel or midfoot are less than their threshold, the toe pressure value is greater than its threshold and the previous phase was FOF or HER.

The foot is considered to be in TOF if the values from all pressure sensors are below their respective threshold and the previous phase was HER or TOF.

For HES, the striking pattern is first evaluated to be either a heel, midfoot, or toe strike. This allows individuals to change how they walk throughout the session as there is no guarantee the individual will strike their foot the same way between gait cycles. For a heel strike, the foot is considered to be in HES when the heel pressure value is greater than or equal to its threshold and the toe is less than or equal to its threshold. For a midfoot strike, the midfoot pressure sensor is greater than or equal to its threshold, and the vertical accelerometer and pitch gyroscope are outside of their respective threshold bounds. For a toe strike, the toe pressure value is greater than or equal to its threshold, and the vertical accelerometer and pitch gyroscope are outside of their respective threshold bounds. For all striking types, HES must be preceded by the TOF or HES phase.

In another embodiment, improvements have been made to the above processes, such that a method for monitoring and evaluating gait, may comprise the following steps:
1. The gait detection algorithm 606 assesses the pressure, gyroscope, and accelerometer values based on thresholds relative to the maximum values observed within this specific batch of data. In certain embodiments, the data are presented to the gait detection algorithm with both feet in the FOF phase.
   i. In certain embodiments, the assessment may be as follows:
      1. TOF events are identified by first identifying mid swing indices, which may comprise locating a local peak gyroscope that is within 20% of the average peak value; filters may be in place to accommodate outliers in magnitude and time.
      2. For each mid swing index, the pre-swing phase may be identified by dynamically altering a threshold value (10-50%) relative to the local peak gyroscope pitch.
      3. The average minimum pressure location across all pressure sensors is used as the TOF event.
2. To identify all other phases, data are first segmented into gait cycles using the TOF events. Preferably, this ensures any failures of the detection algorithm are localized within one gait cycle.

HES events may be identified in two ways: pressure-based, and accelerometer-based.

In a pressure-based embodiment, if a pressure group (heel, midfoot, toes) surpassed the threshold set by the maximum observed pressure, an HES event is triggered.

However, pressure insoles do not always provide reliable data; pressure data can be present when the foot is in swing phase, also termed residual pressure.

In the case of residual pressure at mid swing that surpasses the threshold set for the pressure-based algorithm, a decision-making engine triggers the employment of the accelerometer-based embodiment to evaluate the data.

In the accelerometer-based embodiment, a local minimum value in the forward accelerometer is used to identify the breaking phenomenon found in cyclical gait.

Using average pressure data found in a pre-set time range, which may include for example, 0-100 ms, 100 ms-is, and greater than Is, pressure thresholds may recalibrate and pressure-based algorithm described above is used.

To account for different types of foot planting, HES events may be identified as a heel-strike, mid foot-strike, or a toe-strike. The foot may no longer be identified in the HES event when the vertical accelerometer and pitch gyroscope are within the FOF thresholds.

The foot is considered to be in the FOF phase when the values from the heel, toe, or midfoot pressure sensors are greater than or equal to their threshold, the values of the vertical accelerometer and pitch gyroscope are within their respective bounds (±0.8 G and ±10 degrees/second, respectively), and the previous phase is HES, or FOF.

The foot may be considered to be in HER when the pitch gyroscope is greater than or equal to 20% of the local peak prior to the TOF event.

In certain embodiments, the identified phases of gait are then used for gait assessment. In such embodiments, using the gait cycle phases, a series of temporal (i.e., time-based), spatial (i.e., distance-based), and spatiotemporal (i.e., time and distance-based) variables are calculated for gait assessment. The variables may include both within-foot and between-feet variables. Within-foot variables include stride time, stride length, stride height, swing velocity, stance time/percent, swing time/percent, single support time/percent, and double support time/percent. Between-feet variables include step time, step length, step width, asymmetry, total distance, local dynamic stability, and cadence.

Other sensors embedded in (either permanently or removably), or communicatively coupled to the smart insoles 116 may comprise one or more of: accelerometers, gyroscopes, magnetometers, pressure sensors and ultra-wide bands.

Additionally, the smart insoles 116 may comprise temperature sensors, which may alert a user of a warm up status of the insoles.

Temporal metrics depend on the heel strike and toe-off events, while the spatial metrics (and, by proxy, spatiotemporal metrics) are based on a combination of machine learning algorithms and sensor fusion algorithms to determine the distance travelled within and between feet.

TABLE 1
Gait Metrics Calculated.

| Metric | Unit |
|---|---|
| Gait velocity | km/hr or equivalent |
| Swing velocity | m/s |
| Cadence | steps per minute |
| Stride length | centimetres |
| Step length | centimetres |
| Step height | centimetres |
| Step width | centimetres |
| Walk ratio | mm/step/min |
| Left step duration | seconds |
| Right step duration | seconds |
| Step duration | seconds |
| Stride duration | seconds |
| Single support | % of gait cycle |
| Double support | % of gait cycle |
| Stance phase | % of gait cycle |
| Swing phase | % of gait cycle |
| Single support time | seconds |
| Double support time | seconds |
| Stance time | seconds |
| Swing time | seconds |
| Local dynamic | λ max |
| gait stability | |
| Walking asymmetry | % difference |
| | between feet |

Accordingly, in certain embodiments, gait assessment comprises a determination of one or more of the following: gait velocity, swing velocity, cadence, stride length, step length, step height, step width, walk ratio, left step duration, right step duration, step duration, stride duration, single support, double support, stance phase, swing phase, single support time, double support time, stance time, swing time, local dynamic gait stability, and walking asymmetry.

In certain embodiments, a combination of cut raw signals (pressure, accelerometer, gyroscope, etc.) following activity recognition, gait phases, and spatiotemporal metrics, a pattern classification algorithm identifies the greatest modes of variance within the signal and a machine learning algorithm clusters those features into groups that describe different gait phenotypes (e.g., healthy, ataxic, spastic, hemiplegic, etc.).

An individual may exhibit several phenotypes and express different ones as they fatigue, for example. Accordingly, in certain embodiments, the gait cycles are not grouped as one phenotype. That is, each gait cycle is treated as its own input to the model so an individual can drift into different gait phenotypes over the course of a walking session. In such embodiments, Therefore, the output is a percentage of several phenotypes expressed throughout the session. An example output for a given walking session is 80% hemiplegic, 10% ataxic, and 10% healthy.

In a further embodiment, foot drop events are detected using only raw data signals in a machine learning algorithm. A foot drop is defined as the event following the inability to sustain a dorsiflexed position during the toe-off portion of the gait cycle, resulting in the foot suddenly contacting the floor at heel strike. This is a common symptom experienced by PwMS. In certain embodiments, foot drops are counted and the value added to the database for longitudinal tracking.

In certain embodiments, the systems and methods for monitoring and evaluating a user's gait are conducted in real-time. Such systems and methods may be used for one or more of the following: monitoring gait and/or balance; determining an individual's gait signature; detecting changes in gait patterns; monitoring increased risk of falls; monitoring the progression of diseases and disorders that impact gait, including but not limited include to neurological disorders such as multiple sclerosis (MS) or Parkinson's Disease; assessing the effectiveness of rehabilitation programs; monitoring the post-injury recovery; identifying gait phenotypes and assessing the effect of interventions (including but not limited to physical, medical, psychological, etc.).

In certain embodiments, the system and method of the present disclosure provide healthcare providers and/or pharmaceutical companies and insurers, with insights on conditions which impact gait that would otherwise be undetectable by traditional/existing clinical tools.

The present disclosure provides methods which extract and process data from smart insoles for determining gait and/or balance quality. In certain embodiments, the methods extract and process data from smart insoles comprising one or more sensors selected from a group comprising one or more of: accelerometers, gyroscopes, magnetometers, pressure sensors, ultra-wide band etc and wireless communication capabilities. Other commercially available sensors and/or smart insoles may be employed.

In certain embodiments, the methods of the present disclosure track progression of gait over time using progression algorithms 610. This may be to track disease progression, to assess the effectiveness of rehabilitation programs or monitor post-injury recovery. In certain embodiments, a personalized baseline is calculated using several walking sessions. This is to establish an overall description of their typical walking pattern. A baseline may be calculated at different time points. In this way, interventions may be directly evaluated (i.e., pharmacological, exercise, assistive device, surgical, etc.).

From this baseline, trendlines are calculated to explain how their gait pattern changes (e.g., worsening/improving) over any given timeline (i.e., days, weeks, months, years, etc.). Metrics such as composite movement quality score 276, frequency of assistive device use, number of foot drop events per number of gait cycles, and phenotyping may be used to track progression. In certain embodiments, the composite movement quality score 276 alone or in combination with one or more of the following: frequency of assistive device use, number of foot drop events per number of gait cycles, and phenotyping is used to track progression.

In certain embodiments, the methods of the present disclosure provide feedback to the user and/or healthcare provider. In certain embodiments, the feedback may include alerts including but not limited to increased risk of falls or metrics such as ambulatory index or EDSS. Moreover, visual representations of trendlines may provide clinicians with an early indicator that an intervention is needed to slow the progressing of the disease or change a rehabilitation program is required. Accordingly, in certain embodiments, the methods further comprise changing a patient's treatment and/or rehabilitation regime in response to the feedback provided. In addition, alerts to users regarding increased fall risk may prevent fails by allowing fall prevention measures to be implemented, including strength and balance exercise programs, introduction of assistive walking devices and/or the reduction of safety hazards in the patient's living environment. Accordingly, in certain embodiments, the methods further comprise implementing fall prevention measures in response to the feedback provided.

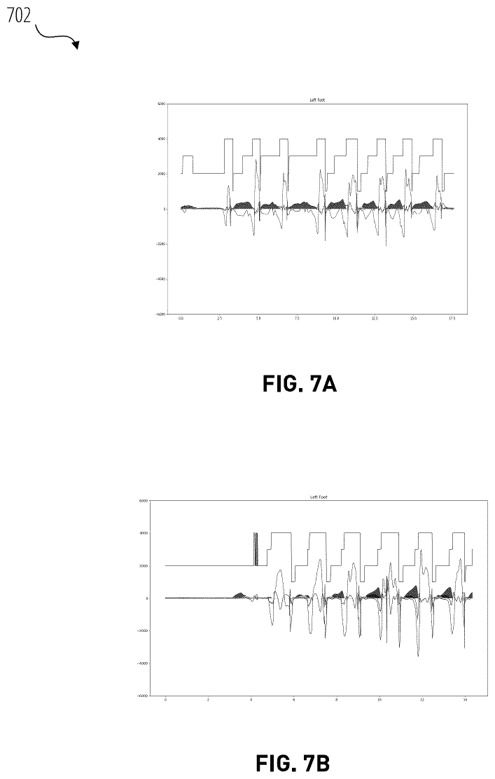

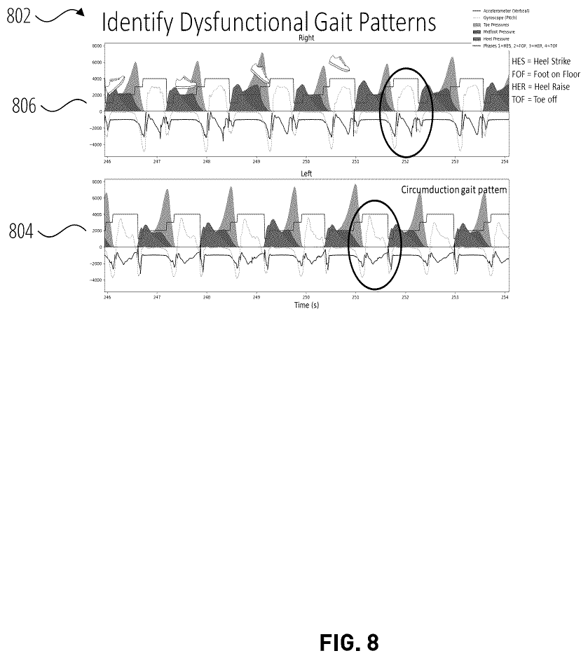

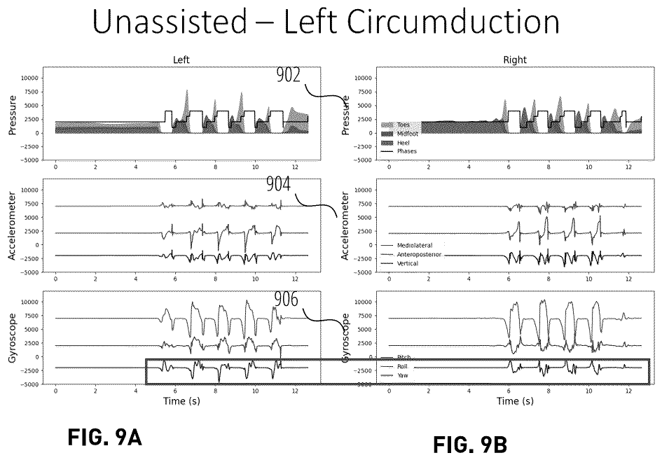

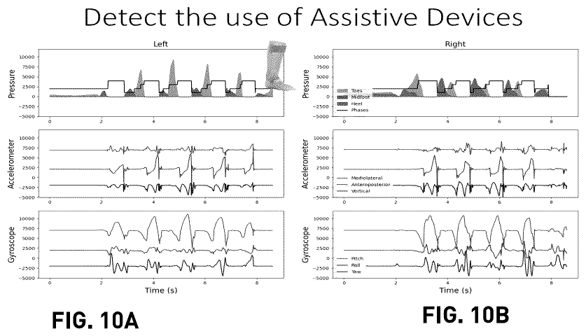

FIG. 7A, FIG. 8, FIGS. 9A &B, FIGS. 10A &B, all illustrate various data processing methods in accordance with one or more aspects of the above methods.

FIG. 7A provides a non-limiting illustrative example of the difference in gait data 702 and/or gait pattern of unassisted vs assisted walking measured with the system of an embodiment of the disclosure

FIG. 7B illustrates a dysfunctional gait pattern measured with the system of an embodiment of the present disclosure. The gait detection method can segment the data into distinct gait cycles, and each gait cycle into the different phases (heel strike, foot on floor, heel raise, and toe off) based on the plantar pressure raw data retrieved from the instrumented insoles.

FIG. 8 illustrates a visual representation of the difference in the patterns of the gyroscope pitch between the left foot 804 (circumduction) and right foot 806 (see area circled).

FIGS. 9A and B illustrates unassisted (left circumduction) gait pattern of an individual whose left leg expresses circumduction (i.e., rotates about the hip to clear their foot as they do not have sufficient dorsi flexion strength) determined using a method in accordance with an embodiment. Sensor measurements may comprise pressure 902, accelerometer 904, and/or gyroscope 906 measurements, among others.

The circled area illustrates difference in patterns of the Yaw gyroscope between the left foot and the right foot. Gait algorithms 606 may detect these differences in order to assess the user's circumduction.

FIG. 10A illustrates ground truth whole-body motion capture, in accordance with an embodiment of the disclosure.

FIG. 10B illustrates assisted gait pattern determined using a method of an embodiment of the present disclosure.

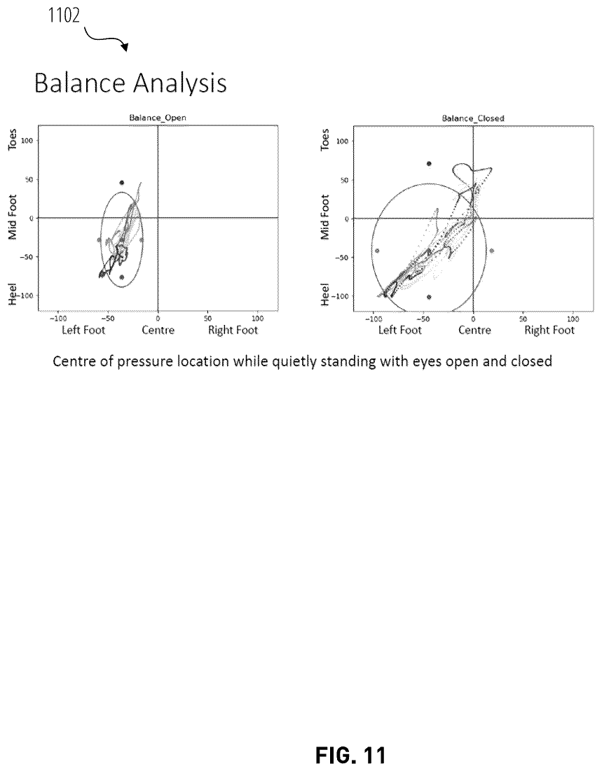

FIG. 11 illustrates graphically analysis of balance data 1102 using a method of an embodiment of the disclosure. Balance metrics can identify changes due to eyes being open and closed (i.e., reducing feedback to the central nervous system to maintain balance). A larger area is indicative of a reduced balance quality.

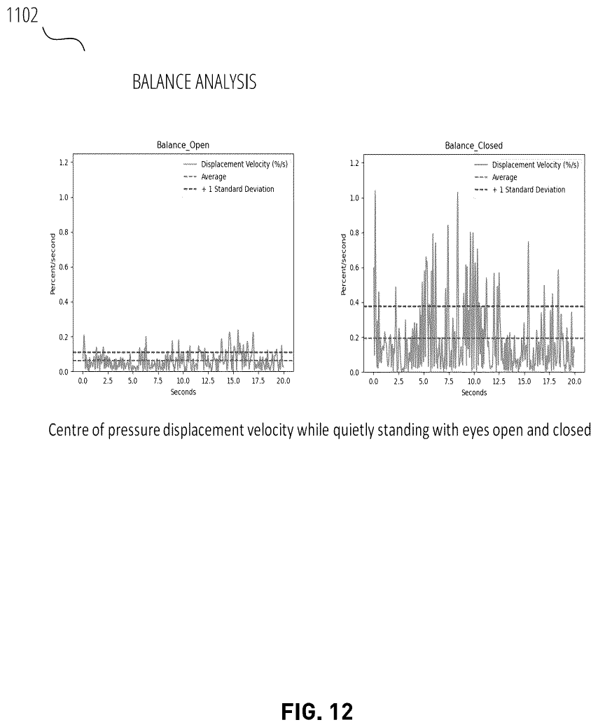

FIG. 12 illustrates analysis of balance data 1102 in accordance with the system of an embodiment of the present disclosure. By extracting and processing the same data as illustrated in FIG. 11, the velocity of the centre of pressure displacement was assessed. Higher values are correlated with an increase in fall risk.

FIG. 13A illustrates a ground truth method 1302 used (MVN Link, Xsens, Netherlands) with a user performing an activity outside of a motion capture laboratory.

Xsens is a commercially available ground truth method.

FIG. 13B illustrates a foot position of an instrumented insole 116. The participant performs a ˜125 metre walk with a stair ascend and descend portion. Foot position was calculated using a Madgwick sensor fusion algorithm.

The fusion method used is widely used in IMU research. The Madgwick filter is a well-known filter for IMUs being used for gait, everyday activities, drones, and cars. The specific update method we use is also standard. It is termed the zero-velocity update method. The zero-velocity update reorients the IMU to gravity and is performed when the foot is on the floor, but can be used any time no movement is detected.

For the CI score, these solutions would not be able to do it on their own out of the box. Xsens would provide users with the information needed (i.e., joint angles, segment positions, rudimentary gait events), but significant independent development is required. Similar for the filtering method, it is only one piece of the puzzle, but an arguably required step.

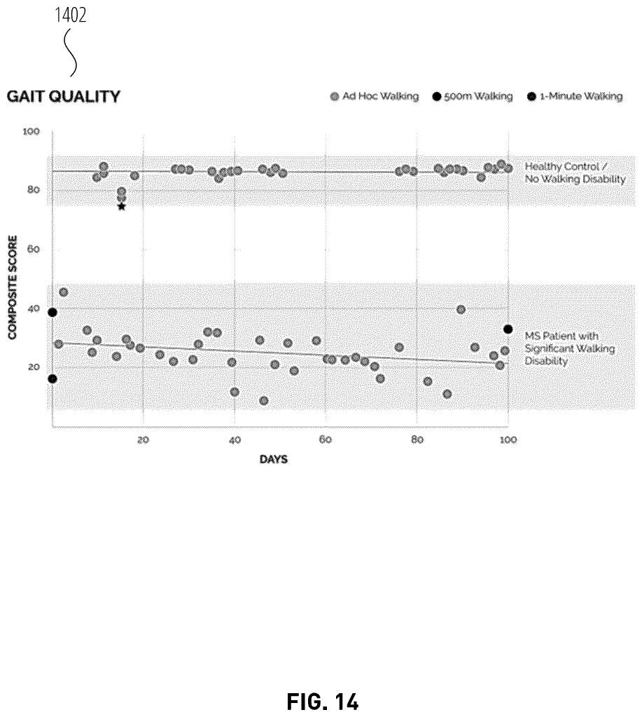

FIG. 14 illustrates provides an illustrative example of a composite score of gait quality calculated over time of an individual, or a graphical composite score progression 1402, in accordance with one embodiment.

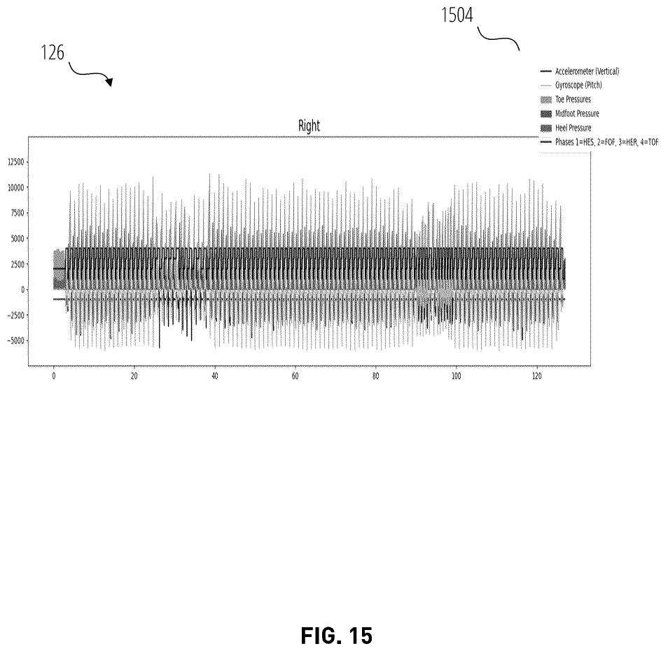

FIG. 15 depicts the raw sensor raw data 126 from the instrumented shoe insole and the gait event overlay of a participant performing an ˜125 metre walk with a stair ascend and descend portion, in accordance with one embodiment. In the illustrated embodiment, the sensor data comprises data measured by vertical accelerometers 904, pitch gyroscopes 906, toe, midfoot, and heel pressure 902 sensors, and sensor-identified events, including: HES, FOF, HER, TOF events.

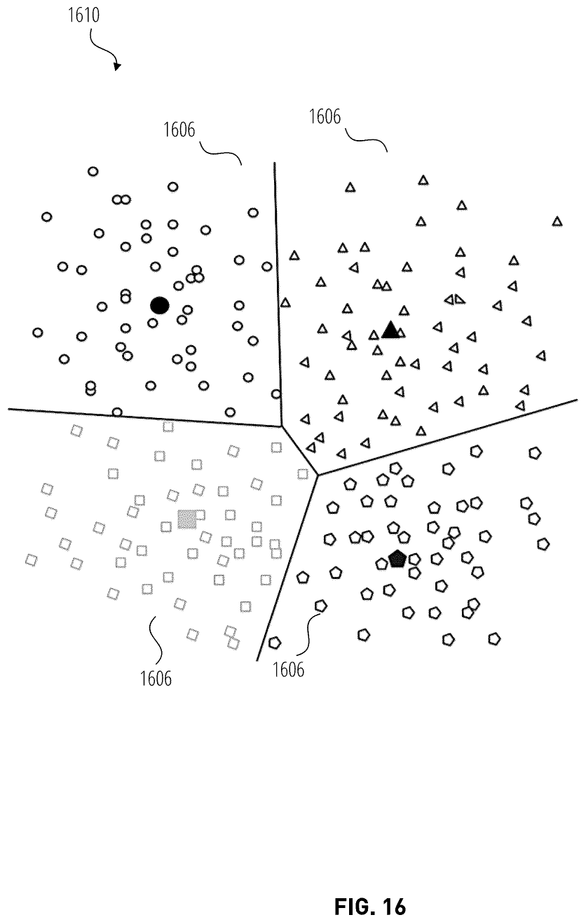

FIG. 16 illustrates depicts a hypothetical machine learning ML clustering 1610 result of principal component scores to identify gait phenotypes 1606 (e.g., healthy, ataxic, hemipelagic, etc.), in accordance with one embodiment.

The skilled person in the art will appreciate that determining phenotypes outside of a laboratory environment is a substantial technical challenge. Known methods typically uses whole-body movement patterns (i.e., lower and upper body motion) to determine gait phenotypes, which is typically restricted to laboratory technology. To analyze the appropriate data (i.e., straight walking, etc.), activity detection needs to be performed, which requires overcoming the technical challenges of training an ML algorithm as discussed. To train an ML algorithm to identify the gait phenotype, ground truth labels from experts who can visually observe the person's walking pattern are also required. Prior to phenotyping, a robust segmentation method is preferably developed to ensure phenotyping is accurate—i.e. walking pattern being segmented into gait events before phenotyping. Further, when moving from a laboratory environment to the wild, the method must have been perfected and tested, before introducing new variables to train the algorithms on. The present disclosure advantageously overcomes those technical challenges by only using, in accordance with different embodiments, foot motion to determine gait phenotypes.

Phenotyping in previous literature is also described as spatiotemporal (e.g., stride time, stride length, etc.) and whole-body kinematic changes (i.e., joint angles) compared to healthy controls. They measure the person for several seconds on specialized pressure maps, using proprietary accelerometer software, and expensive motion capture systems, that are restricted to laboratories.

In the lab, only short segments of data can be measured while a user is moving overground. The present method assesses data over many consecutive these segments to improve its accuracy, which in turn introduces new challenges in that the methods need to be sensitive enough to detect changes in gait over a long period of time.

These challenges may be overcome by generating labels in a lab, extensively testing them, and then employing the solution in the wild, iteratively evaluating phenotype progression over time.

Phenotyping gait by coupling smart insole data with novel algorithms employs a practical method for assessing MS patients' gaits.

In certain embodiments, the raw signals are first organized using the gait phases, and is analyzed using principal component analysis (PCA) to identify the modes of variability in that signal, deemed principal components (PCs). Scores are given to each gait cycle that relates to how well a given PC explains the raw data within that gait cycle. These PC scores are then input to a clustering-based machine learning algorithm (e.g., K-means, hierarchical cluster analysis). Using ground truth labels which have been previously created from neurologists analyzing videos of people with multiple sclerosis (PwMS), the clusters that are created by the machine learning algorithm are labelled as a specific gait phenotype.

Employing the disclosed gait phenotyping methods, gait insights 220 may be delivered using terminology that is meaningful to clinicians. For example, an assessment describing a patient's mild spasticity in left leg progressing to moderate/high spasticity over a time period. Insights 220 may be geared selectively towards patients, by converting the phenotypes 1606 into challenges that patients will face. For example, an assessment describing that a patient may struggle to walk for more than 5 minutes.

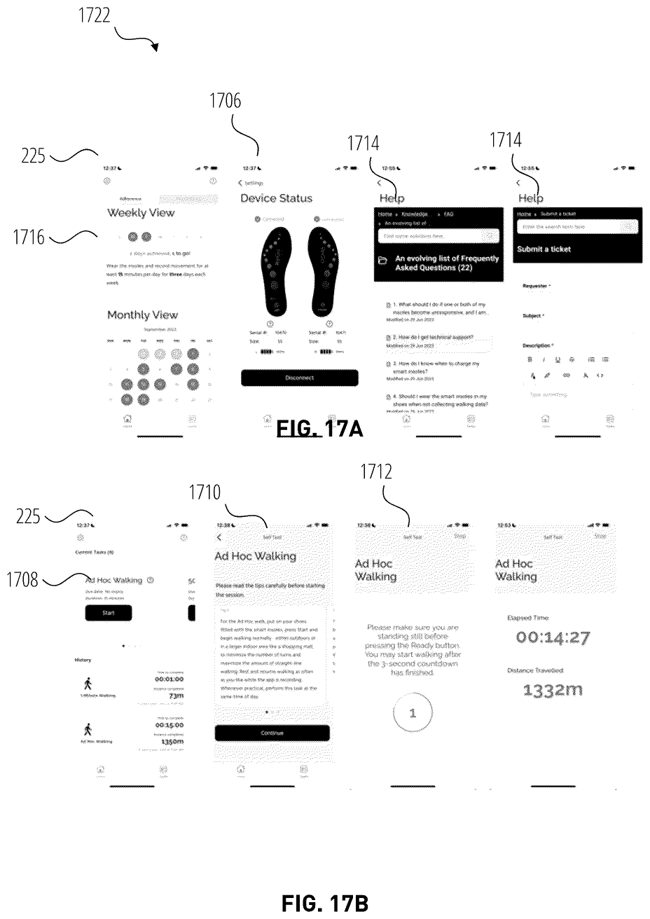

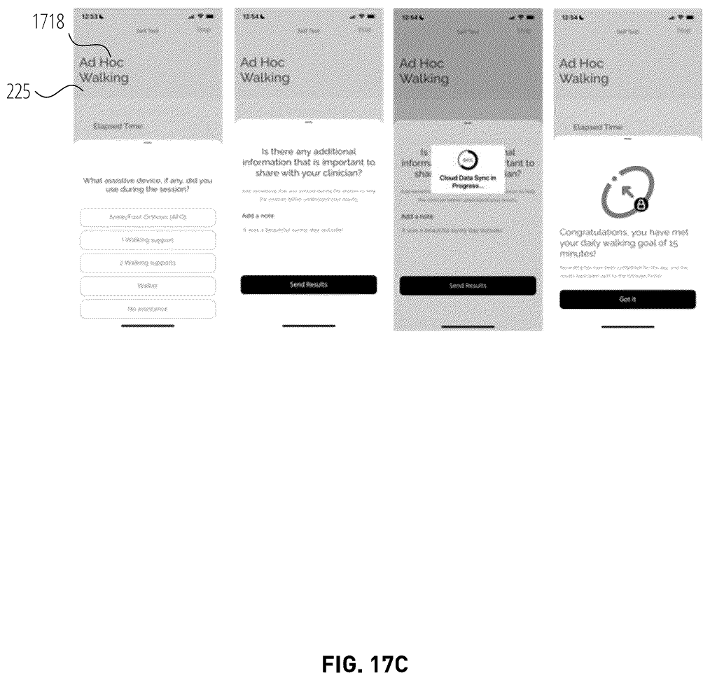

FIG. 17A and FIG. 17B and FIG. 17C, generally, illustrate one or more steps set forth by a user interface 225 of a mobile app 1722 of an embodiment of the present disclosure.

The present disclosure further comprises a system comprising a computing device configured to perform the method steps set forth above. In certain embodiments, the computing device and any databases are cloud-based.

Also provided is a non-transitory, computer readable memory and/or medium having recorded thereon statements and instructions for execution by a computer the method steps detailed above.

In the patient environment 404, as was illustrated in FIG. 4, the user interface 225 of the mobile app 1722 may display one or more interactive screen displays, such that a user may view scheduled exercises, progress, processed data, as well as device settings.

The one or more steps executed by the app 1722 may comprise set-up steps, including but not limited to pairing Bluetooth enables insoles with the app, setting up a user profile with user data (age, weight, height, etc.), and setting up a clinician profile (electronic medical records, goals which may be sent to the user, and the like). The one or more steps may comprise prompts to the user (walk, stand, balance, etc.) or one or more insights to the clinician.

FIG. 17A illustrates a user interface 225 for adherence tracking 1716 by the 1722.

The mobile app 1722 may tracks adherence to a walking goal, which may be set by a clinician or by the user.

The mobile app 1722 may include a device status (size, serial number, battery level) page with a built-in help page 1714, which may, upon a user selecting the help function, trigger a mechanism alerting a need for technical support. The help page may further display frequently asked questions (FAQ).

Clinicians may set, on the clinician portal 414, expectations for patient exercise, which may then be sent, over a network 136, to the patient environment 404, and viewed on the adherence tracking 1716 page by the patient. Patients may record their exercise data in the adherence environment, by pairing their smart insoles 116 with their mobile device and the mobile app, where it may be sent, automatically or manually, to the clinician portal 414, over a network.

Still further, the mobile app 1722 may first send the patient data to the cloud-based computing device 152, where the plurality of algorithms as illustrated in FIGS. 1 and 2. May process the data and assign a composite score 276 to the data, before sending the score 276 to the clinician portal.

Preferably, but not necessarily, a step triggering a reminder to the patient to conduct an exercise, occurs automatically after a pre-determined (either by the clinician or by an ML algorithm 144), and the steps sending the data to the cloud, processing the raw data, and sending the score to the clinician portal, and occur automatically once the exercise is performed, thereby minimizing the clinician's time spent sending reminders.

FIG. 17B illustrates an aspect of a user interface 225 in which the mobile app 1722 displays interactive gait-test prompts 1708. In addition to ad-hoc walking, patient's may be asked to perform various gait-tests.

Clinicians may set, on the clinician portal 414, expectations for patient exercise, which may then be sent, over a network 136, to the patient environment 404, and viewed on the adherence tracking 1716 page by the patient. Patients may record their exercise data in the adherence environment, by pairing their smart insoles 116 with their mobile device and the mobile app, where it may be sent, automatically or manually, to the clinician portal 414, over a network.

Still further, the mobile app 1722 may first send the patient data to the cloud-based computing device 152, where the plurality of algorithms as illustrated in FIGS. 1 and 2. May process the data and assign a composite score 276 to the data, before sending the score 276 to the clinician portal.

FIG. 17C illustrates an aspect of a user interface 225 in which the mobile app 1722 displays interactive supplemental gait-test information 1718. Patients may be prompted, after completing an exercise or a gait test, to answer some supplemental questions, which may include, but are not limited to, assistive walking devices used, additional notes to send to the clinician, identifying from a selectable dropdown list further symptoms the patient is experiencing, and the like.

The data obtained walking prompts, additional gait-test prompts, and supplemental questions may be sent to the cloud-based computing device 152 and processed by the one or more algorithms before sending the score 276 to the clinician portal.

Any of the processed results may be sent to the user patient environment 404 of the system as well.

The mobile app 1722 may further comprise a prompt for the clinician prior to releasing the processed patient data to the patient environment 404.

Requests may be triggered by the mobile app 1722 after periods of inactivity, or triggered by missing or incomplete data identified by one or more machine learning algorithms before completing an assessment or a time-based (progression) assessment of the patient's composite movement quality score 276.

FIG. 18 displays, graphically, correlation results comparing CI 276 and EDSS 1828 to MSWS12 questions 1824.

Extended Disability Status Scale (EDSS 1828) represents a global standard for MS neurologist patient assessments. Each vertical line represents a different trial participant.

MSWS-12 1824 is a clinically validated Patient Reported Outcome (PRO) measure of MS walking quality.

For each of the CI 276 and the EDSS 1828, results for Spearman Rho 1822 were plotted against each MSWS12 question 1824.

x marks 1830 represent a significance threshold of p<0.05, triangle marks 1836 represent a significance threshold of p<0.01, and circle marks 1838 represent an acceptance of the null hypothesis. Absolute values for rho are used for visualization; all CI rho values are negative.

The composite index score 276 as illustrated, is closely aligned with the MSWS-12 assessment. The primary outlier (out of 40 participants) in the illustrated graph may be attributed to the heightened sensitivity of the score 276 to balance impairments, the score 276 identifying balance impairments not captured by present models (such as MSWS-12).

FIG. 19 depicts, graphically, the CI scores calculated for each participant during an indoor 500 metre walk test.

In FIG. 19, CI scores 276 are sorted based on their Extended Disability Status Scale (EDSS 1828). Boxplots drawn in Blue represents healthy participants 1902, Green represents an EDSS score <3.5 1904 (low disability level), Yellow represents 3.5<EDSS >4.5 1908 (medium disability level), and Red represents an EDSS >4.5. EDSS 1906 (high disability level).

In the illustrated examples, the presented framework uses raw pressure, accelerometer, and gyroscope data streamed to a mobile device 264 to analyze the gait patterns of PwMS.

Through one or more ML algorithms 146 (including group 1 algorithms 248, group 2 algorithms 244, group 3 algorithms 240, group 4 algorithms 242, group 5 algorithms 246), raw data 126 is segmented into activity type, gait cycles, and the like, and analyzed to produce a plurality gait metrics.

These gait metrics are further processed by the group 6 algorithm 250 in order to conclusively assign a composite score 276, as illustrated in FIG. 19 to be a single overarching metric termed the composite movement quality score 276, or composite index 276 (CI), to provide useful and actionable insights to patients and clinicians. The CI 276 may be displayed on a mobile device 264 or a computing device 112, in a graphical user interfaces 225.

The CI 276 may be trained by a support vector machine (SVM) in order to classify healthy participants and PwMS; the participant's projected location on the hyperplane calculates a Z-score, translated to a value between 0-100%.

Preferably, the SVM measures and analyzes a plurality of features relating to a user's mobility. In the Example 3. Below, the chosen SVM uses 22 features and achieves an accuracy, —F1-score and κ of 95.58%, 95.59%, and 0.909, respectively, suggesting that the trained SVM model is highly accurate and reliable for classifying the tested population.

Known methods have not, thus far, been capable of developing a framework for a composite index, interpretable by clinicians and patients, in part because they did not incorporate means of evaluating healthy patients as well as PwMS.

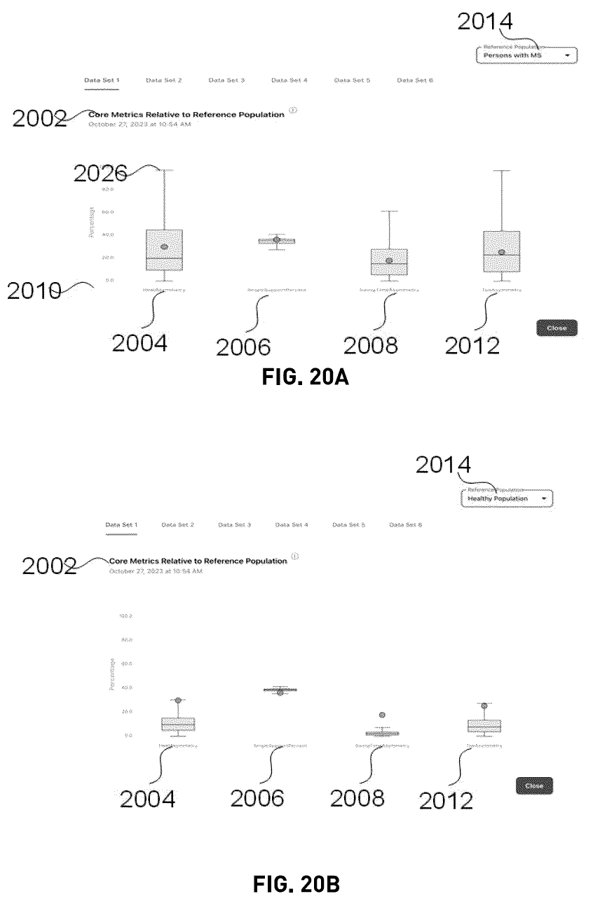

The features of the model in the illustrated embodiment are from all major gait domains as reported by several research endeavors: pace, rhythm, asymmetry, and variability (e.g., Kushioka et al., 2022; Lord et al., 2014; Monaghan et al., 2021; Shema-Shiratzky et al., 2019), and can be seen in greater detail in FIG. 20A and FIG. 20B.

It can be seen by way of the examples below, that the composite score 276 of the present disclosure exhibits a higher ML performance metrics than others previously presented (e.g., Trentzsch et al., 2021).

Future iterations of the composite index 276 may comprise tuning the one or more algorithms for processing the data received by group 6 algorithm: composite score 250, to be sensitive to identifying diseases or disease states.

Future iterations of the composite index 276 may comprise training the CI 276 with data from a multisite project, in order to capture weekly walking patterns of PwMS over several months.

As the CI evolves, more/different features may be included in the SVM model to improve accuracy of the assessments.

Measuring gait quality in PwMS with a CI 276 produces an objective outcome measure that is very strongly correlated to accepted disability metrics.

The CI score 276 can be used to determine trends in gait quality but also as a starting point to investigate which metrics are causing the CI to increase/decrease (e.g., increase in double support time, reduction in cadence), an opportunity not available to traditional walking tests that only measure time and distance.

Further, a wearable solution to assess gait quality removes several barriers for PwMS when interacting with the healthcare system, allowing for remote monitoring and an increased frequency in gait evaluations, allowing for a healthcare approach that can be proactive rather than reactive.

In accordance with an embodiment of the disclosure, gait algorithms 606 may be modified to be robust to residual pressure (i.e., significant pressure when the foot is off the ground). Modifications may comprise a decision matrix which first identifies toe-offs using a foot pitch (flexion-extension) gyroscope 906, then deciding whether to use an unmodified gait algorithm 606 if there is no residual pressure or identifying the breaking phenomenon in the forward accelerometer as the heel strike, then using the unmodified gait algorithms 606 for all other aspects. Modifying the gait algorithm to be able to identify toe-off may significantly improve the performance of the methods in real-life use.

In accordance with another embodiment of the disclosure, the composite movement quality score 276 or CI may be converted into a prediction of a patient's EDSS 1828, within specific ranges (low/moderate/high) as well as their MSWS-12 score.

To gain a better understanding of the disclosure described herein, the following examples are set forth. It will be understood that these examples are intended to describe illustrative embodiments of the disclosure and are not intended to limit the scope of the disclosure in any way.

### EXAMPLES

### Example 1: Comparison of Results Obtained Using the System and Method of the Present Disclosure Compared to a Markerless Motion Capture System

Ten healthy individuals with no current musculoskeletal injury or neurological disorder were recruited to perform 39 overground walks on a six-metre walkway with two embedded force plates (FP-4060, Bertec, USA) at their preferred walking speed. Participants wore a pair of instrumented insoles with pressure, accelerometer and gyroscope sensors (Neurogait 3.0, Salted, South Korea) while their whole-body kinematics were simultaneously collected using eight video cameras (Vue, Vicon, UK). The video camera data were processed using Theia 3D (C-motion, USA) to identify joint landmarks and Visual 3D (C-motion, USA) was used to automatically identify gait events (heel strike and toe off) based on kinematics and force plate data (all events were visually identified and adjusted as necessary). Instrumented shoe insole data were analyzed using the methods described in the present application. That is, gait events were segmented into heel strike, foot on floor, heel raise, and toe off events based on pressure, accelerometer, and gyroscope thresholds and logical events. Using the gait events from each technology, the same temporal variables were calculated and compared using a 3,1 intraclass correlation (ICC_{(3,1)}) in Python. A total of 1206 gait cycles were used for each comparison. An average ICC_{(3,1)} value of 0.955 was calculated across all metrics representing an excellent reliability between motion capture systems. Results averaged between left and right side are presented in Table 2.

TABLE 2
Instrumented Shoe Insole Validation Compared
to a Markerless Motion Capture System

| Metric | ICC_{(3, 1)} | CI95% |
|---|---|---|
| Stride Time | 0.999 | [0.995, 1.000] |
| Stance Time | 0.992 | [0.970, 1.000] |
| Swing Time | 0.984 | [0.935, 0.995] |
| Stance Percent | 0.911 | [0.690, 0.980] |
| Swing Percent | 0.911 | [0.690, 0.980] |
| Single Support Time | 0.982 | [0.935, 1.000] |
| Double Support Time | 0.935 | [0.760, 0.980] |
| Single Support Percent | 0.925 | [0.730, 0.980] |
| Double Support Percent | 0.918 | [0.705, 0.980] |
| Step Time | 0.999 | [1.000, 1.000] |
| Cadence | 0.989 | [0.960, 1.000] |

Note: Values are averaged between left and right sides. The markerless motion capture system used is Theia 3D (c-motion, USA). Percent represents the percentage of gait cycle from heel strike to heel strike of the same foot. ICC_{(3,1)} represents an intraclass correlation coefficient of fixed raters where those raters are the only of interest. CI95% represents 95% confidence intervals of the ICCs. ICC thresholds: poor: <0.50, moderate: 0.50-0.75, good: 0.75-0.90, and excellent: >0.90.

### Example 2: Temporal and Spatial Metrics of Unassisted Vs Assisted Gait

A case study a participant with multiple sclerosis (PwMS) who was recruited for an initial investigation is described here. Specifically, a sub-portion of our protocol where individuals were asked to walk over a six-metre walkway with two embedded force plates (FP-4060, Bertec, USA) at their preferred walking speed is presented. The same methods as example 1 were used here. That is, video camera and instrumented insole data were simultaneously collected and their respective processing steps were performed to obtain gait events and thus calculate temporal variables. However, this example focuses on the results obtained from the instrumented insoles.

The participant of interest arrived at the laboratory with an ankle foot orthosis (AFO; unilateral left side) and a cane (unilateral right side). The individual was asked to perform the overground walk six times with two repetitions of unassisted, assisted with an AFO, and assisted with AFO+cane, respectively.

It is demonstrated in Table 3. below how methods of the present disclosure have the ability to detect how an assistive device can alters an individual's gait pattern. Notably for this case, the individual exhibited a decrease in variability (i.e., lower standard deviations) for the majority of temporal gait metrics.

TABLE 3
Objective Difference Between Conditions
Objective Differences Between Conditions
Temporal Metrics

| | | | Double | Single | | | Stance | Swing | |
|---|---|---|---|---|---|---|---|---|---|
| | | Cadence | Support | Support | Stance | Swing | Percent | Percent | Step |
| | Asymmetry | (step/min) | Time (s) | Time (s) | Time (s) | Time (s) | (%) | (%) | Time (s) |
| Unassisted | 7.12 | 94.87 (13.4) | 0.23 (0.03) | 0.52 (0.07) | 0.75 (0.08) | 0.50 (0.09) | 53.8 (4.76) | 40.3 (4.76) | 0.64 (0.09) |
| AFD | 4.52 | 90.57 (7.58) | 0.22 (0.04) | 0.58 (0.07) | 0.80 (0.04) | 0.52 (0.04) | 60.3 (2.41) | 39.7 (2.41) | 0.60 (0.06) |
| AFO + Cane | 1.05 | 95.49 (7.44) | 0.16 (0.06) | 0.58 (0.06) | 0.74 (0.02) | 0.54 (0.01) | 57.5 (0.64) | 42.0 (0.64) | 0.60 (0.05) |

AFO: Ankle Foot Orthosis, 1 cane used unilaterally

### Example 3. Composite Movement Quality Score Development

In this example, a study evaluating the effectiveness of developing a framework for a composite movement quality score 276 is disclosed.

Significant technical challenges are present in the development of such a framework because laboratory data is required to ensure the spatiotemporal (or any variable) used to train a machine learning (ML) algorithm is valid

Good-quality labels are needed when training ML algorithms. A large enough sample size is also needed. In the illustrated example, a clinician provided participants' disability status, and their perceived walking ability through a survey (MSWS12).

The best decisions need to be made in terms of feature selection, hyperparameters, and ML architecture.

As disclosed below, the example overcomes these challenges by: monitoring both healthy individuals and PwMS to determine 22 key metrics in evaluating and determining a composite index. The example illustrates a projection of participants onto the decision boundary, produce a Z score from and expressed as a 0-100%.

Participants: 22 healthy and 19 PwMS were recruited for this investigation. Healthy was defined as any individual who has not experienced a musculoskeletal injury within the last six months at the time of testing and does not suffer from a neurological disorder. PwMS were recruited from The Ottawa Hospital, who had undergone a neurological exam within the last 12 months, where they were given an EDSS score of less than 7.0; the average EDSS score was 3.76 (±1.71). Demographic information for all participants is displayed in Table 4.

TABLE 4
Participant Demographics

| Population | Age | EDSS |
|---|---|---|
| Healthy | 28.1 | — |
| | (6.66) | |
| PwMS | 54.2 | 3.76 |
| | (16.5) | (1.71) |

### Movement Protocol and Instrumentation:

The original insole inside the participant's shoe was replaced with an instrumented shoe insole 116, (in the present example, the insole was an off-the shelf insole from ReGo, Moticon, Germany; 50 Hz), which streamed raw pressure, accelerometer, and gyroscope data to a mobile application (developed by Celestra Health, Canada).

If the shoe insole could not be removed, the instrumented insole was placed on top of the existing shoe insole. Participants were asked to perform walking tasks in the motion capture laboratory (i.e., overground and treadmill walks), in the indoor hallways outside the laboratory (500- and 125-metre walks), and outdoors on a paved multiuse pathway (500-metre walk; healthy only).

In the laboratory, all participants performed at least six overground walks measuring six metres over two force plates (FP-4060, Bertec, USA; 1000 Hz).

If participants arrived with an assistive device, the six walks were split evenly to progress the participant from unassisted to fully assisted (e.g., a participant with a cane had three repetitions unassisted and three with a cane).

All healthy (optionally PwMS) performed a seven-minute walk on a treadmill at their preferred walking speed. Whole-body kinematics were collected using an eight-video-camera system (Vue, Vicon, UK; 50 Hz) and analyzed using Theia3D (Theia, USA).

In the hallways adjacent to the laboratory, participants were asked to walk up to 500 metres around two pylons placed 25 metres apart, making left- and right-hand turns around the pylons (i.e., making a FIG. 8). Participants were instructed to walk comfortably without stopping; chairs were set up along the hallway to provide rest if needed, and a research assistant walked with participants at higher disability levels.

Participants were encouraged to perform the 500-metre walk without assistance; however, they were not prevented from using an assistive device if requested. All healthy (optionally PwMS) also performed a 125-metre walk indoors, which included several turns (left and right), a stair ascend, and a stair descend section. Only healthy participants performed a 500-metre walk outside, which contained four right-hand turns and a slight uphill and downhill section on a paved multiuse pathway.

### Data Analysis

### Markerless Motion Capture Data

For the movements performed in the laboratory, force platform data and kinematics calculated through Theia 3D were imported into Visual3D (C-motion, USA). Gait events were initially identified using the automatic gait detection pipeline and imported into Vicon Nexus 2 (Vicon, UK), where they were visually verified using video data and manually adjusted as necessary. Gait event and spatial data were then exported to Matlab (MathWorks, USA) and used to calculate spatiotemporal variables.

### Instrumented Insole Data

Instrumented insole data were streamed via Bluetooth to the Celestra Health mobile application on a mobile device 264. Raw pressure, accelerometer, and gyroscope data were exported to a .csv file, which was imported into Python 3.10.

A human activity recognition (HAR) layer with a fully connected neural network (NN) architecture identified standing, walking, turning, stair ascend, and stair descend activities. The raw pressure, accelerometer, and gyroscope data were passed into an NN with a softmax decision layer with a logical layer to filter activities with short window sizes. For the present example, only the walking activity label is discussed.

Following HAR, data identified as walking passed through a gait detection algorithm developed based on the work of Chatzaki et al. (2021). In short, the pressure sensors were grouped into heel, midfoot, and toe segments and the developed algorithm used threshold values (i.e., 7% maximum pressure) to trigger different gait events based on the previous state parameter: heel strike (HES), foot-on-floor (FOF), heel raise (HER) and toe off (TOF).

As acknowledged by Chatzaki et al. (2021), individuals may not strike the floor with their heel to trigger an HES event; therefore, the gait detection algorithm accommodates HES events triggered by a toe strike.

Building on the work done by Chatzaki et al. (2021), an embodiment of the algorithm of the present disclosure includes a midfoot pressure group, which allows for HES events to be triggered by midfoot-strike events.

Still further, in another embodiment, the algorithm may comprise a midfoot pressure group combined with stricter parameters were also set for FOF events: vertical acceleration has to be between 1.1 and 0.9 m/s², and pitch gyroscope had to be between 20 and −5 deg/s. These additional parameters may be used to aid in the reliability of the FOF event, as this event is critical to the performance of the sensor fusion algorithm discussed below.

In another embodiment, an algorithm of the present disclosure combines one or more of: pressure groups, strict FOF parameters, and IMU measurements. The resulting algorithm results in highly accurate and robust assessments and evaluations of gait in the wild.

Following gait detection, the accelerometer and the gyroscope data are fused using a Madgwick filter to calculate the position of the insoles from an arbitrary starting position using methods described by (Rebula et al., 2013).

Participants are instructed to stand for three seconds for all trials, which was used to determine and remove signal biases from all IMU channels. Following bias removal, the accelerometer was rotated to an earth reference frame, and strict FOF periods were identified. These FOF periods were calculated by first high-pass filtering (cut off 0.001 Hz) and then low-pass filtering (cut off 5 Hz) the Euclidean norm of the accelerometer; subindices of the FOF segment that fell below 1.5 times the median absolute deviation of that FOF segment was used as periods of zero velocity to update the orientation of the fusion algorithm. Initial convergence of the Madgwick filter was done over the middle 75% of the three-second stationary period using a gain of 0.057; this gain value was used for each strict FOF period, while a gain of 0.018 was used during all other phases.

As per Rebula et al. (2013), the orientation was used to rotate the accelerometer within the earth's reference frame and was integrated to calculate velocity using the trapezoidal method. At each period of strict FOF, velocity was set to 0 m/s and the data were detrended between each FOF event (Rebula et al., 2013). The corrected velocity was then integrated to calculate the insole's position over time.

For standardization, walking data were split into 10-second segments to ensure that central tendency measures from minutes-long data weren't being compared to those from seconds. For each 10-second segment, 15 spatiotemporal gait metrics (e.g., step time, stride time, swing time, stance time, single and double support time, etc.), six pressure metrics (i.e., the average and average peak area under the curve during stance phase for each pressure group), six IMU metrics (i.e., average peak, and range, root mean squared error, and meanSD for the accelerometer and gyroscope), and their respective variability metrics when appropriate (i.e., asymmetry, and standard deviation), resulting in 128 gait metrics. When appropriate, each metric's average and standard deviation between feet and across the 10-second segment were calculated, resulting in 90 metrics.

Although segments are 10 seconds in the above example, it should be readily understood that the data may be segmented into shorter, segments, i.e. 1-10 second segments, and longer, i.e. 10-120 second segments, and still further, the segments may be shorter than 1 second and longer than 120 seconds.

### Composite Index (CI): Training

A support vector machine (SVM) was trained to classify the gait metrics of healthy participants and PwMS. The training data consisted of 1743 rows (899 healthy; 843 PwMS) of 90 gait metrics representing 10-second walking segments retrieved from all walking trials. Feature selection was performed to reduce the feature space by removing redundant or irrelevant features in three steps: 1) T-tests identified 84 metrics as significantly different (p<0.05) between groups; 2) Pearson correlations were run, and those metrics that had an absolute r value ≥0.99 were removed, resulting in 69 metrics; and 3) an iterative partial least squares (PLS) regression was used to iteratively identify which subset of features best separated the class labels (i.e., incrementally adding one feature up to 69 features). An SVM was trained using each subset of features identified through PLS. For each feature set, features were scaled using sklearn's standard scaler (Pedregosa et al., 2011) and the SVM's hyperparameters were optimized using a grid search and cross-validated using a leave-one-out approach, where entire participants' data were iteratively removed; assessed hyperparameters are listed in Table 5.

TABLE 5
Hyperparameters

| Parameter | Value |
|---|---|
| C | 0.1, 1, 10 |
| Gamma | 0.001, 0.01, 0.1, 1 |
| Kernal | Linear, radial basis |
| | function, polynomial |

Note:
grid search was performed using sklearn's GridSearchCV method. Bolded values are the hyperparameters used for the final model.

For each PLS iteration, the best hyperparameters from the grid search operation were selected based on accuracy and F1, resulting in 69 trained SVM models to classify PwMS and healthy participants.

CI values were calculated for all participants across all 69 SVM models. To calculate the CI, the training data was projected onto the trained SVM's hyperplane to calculate its mean and standard deviation. Using the mean and standard deviation of the projected training data, the Z-score of a participant's projected data is calculated. To receive a score of 0-100%, the cumulative distribution function was applied to the Z-score. To select the final SVM model, the accuracy, F1-score, and correlation values (Pearson or Spearman used when appropriate) comparing the CI scores to the PwMS's EDSS and MSWS12 scores and all participant's cumulative Z-score across all metrics (i.e., Z-scores were calculated for each metric and averaged then compared to CI) were evaluated.

### Results: Accuracy and Correlation

Spearman Rho (ρ) values were interpreted as very strong (ρ>0.69), strong (0.40<ρ≤0.69), moderate (0.29<ρ≤0.40), weak (0.19<ρ≤0.29), and negligible (0<ρ≤0.19). The significant cut-off to reject the null hypothesis was set to p<0.05; if p<0.01, the results were interpreted as highly significant. The SVM's accuracy, F1, and Cohen's Kappa (κ) score were calculated using the sklearn library.

### Composite Index (276)

An SVM with 22 features (Table 6) was chosen to classify Healthy Controls vs. PwMS. using a linear kernal with a regularization parameter (C) of 0.1 and a gamma or 0.001; the model is 95.58% accurate with an F1-score of 95.59% and a κ of 0.909.

TABLE 6
Gait Metrics Chosen for SVM

| Gait Metric | Gait Metric Domain |
|---|---|
| Swing Phase Percent Asymmetry | Asymmetry |
| Toe Pressure Asymmetry | |
| Heel Pressure Asymmetry | |
| Distance travelled in 10 seconds | Pace |
| Cadence (steps/minute) | |
| MeanSD Accelerometer Euclidean norm | Movement variability |
| MeanSD Gyroscope Euclidean norm | |
| RMSE Accelerometer Euclidean norm | |
| RMSE Gyroscope Euclidean norm | |
| SD of Peak Heel Pressure | Pressure variability |
| MeanSD Toe Pressure | |
| SD of Right foot's Percent | |
| Toe pressure | |
| MeanSD Heel Pressure | |
| MeanSD Midfoot Pressure | |
| RMSE Midfoot pressure across steps | |
| Percent time Single Support | Rhythm |
| Double Support Time (seconds) | |
| Stance Time (seconds) | |
| Average Peak Midfoot Pressure | |
| Step Time (seconds) | |
| Average Peak Accelerometer | |
| Euclidean Norm | |
| Average Peak Gyroscope Euclidean | |
| Norm | |

Note: Gait metrics in the present example were averaged across sides of the body unless otherwise stated. RMSE, SD, MeanSD, and average peak, are calculated using gait cycles within each 10 second walking bout. RMSE=root mean squared error; SD=standard deviation, MeanSD=average standard deviation across all gait cycles for each normalized time point.

The CI 276 values had a very strong significant negative correlation with the EDSS score: ρ=−0.805 (p<0.001). In relation to the MSWS12 scores, the CI values had ten significant negative correlations (seven were highly significant) that ranged from strong to very strong (i.e., ρ>0.40); on average across all questions, ρ=−0.602 (±0.154; range: −0.343-−0.807).

Similarly, in relation to the MSWS12 scores, EDSS had nine significant positive correlations (seven were highly significant); on average across all questions, p=0.550 (±0.166; range: −0.246-−0.789).

When MSWS-12 results are expressed as a single value (i.e., the sum of all answers), EDSS was significantly and strongly positively correlated (ρ=0.688, p=0.001), and CI was significantly and very strongly negatively correlated (ρ=−0.721, p<0.001).

The present example included young-healthy individuals in the dataset, as well as PwMS at high disability levels, in order to develop a comprehensive CI score. Different disease states and an “ideal” walking pattern were necessary to understand the distribution of walking ability.

The resulting CI 276 had a very strong negative correlation compared to the widely accepted EDSS score (ρ=−0.805) and had superior correlation values than EDSS across all MSWS12 questions (CI: ρ=−0.602±0.154; EDSS: ρ=−0.550±0.166), the CI followed similar trends to the EDSS across all MSWS12 questions with an average absolute difference between correlation coefficients being 0.091 (±0.060), and when MSWS12 is represented as a single value, the EDSS and CI scores had strong to very strong correlations (EDSS: ρ=0.688; CI: ρ=−0.721). The correlations found in the present study are similar in significance albeit lower for the correlation coefficient to previous research correlating EDSS and MSWS12 (ρ=0.78 p=0.0001; Motl and Snook, 2008), likely driven by the considerable differences in population size (19 PwMS vs 132).

The present disclosure illustrates, significantly, that MSWS12 and EDSS may be excellent predictors of one another, while the MSWS12 provides much-needed context to perceived mobility dysfunctions.

Further, the CI's very strong correlations to EDSS and MSWS12 display its utility for objectively quantifying a PwMS's gait quality while not confining patients to burdensome clinical visits.

FIG. 20A and FIG. 20B illustrate an aspect of a user interface for displaying a composite movement quality score 276 platform, trained on a combination of metrics, to identify core metrics pertaining to gait data from reference populations 2014, in accordance with an embodiment.

FIG. 20A illustrates core four core metrics associated with the reference population 2014 being persons with MS, while FIG. 20B illustrates four core metrics associated with a reference population 2014 being a healthy control population.

This information may be displayed in a clinician portal 414 of a system according to an aspect of the disclosure. Clinicians may select, in the user interface 225 of the clinician portal 414, a given composite index walking score 276 to view underlying metrics and how they compare with both healthy population and MS populations. The interactive platform transforms the raw data to a tangible score, without losing the underlying metrics, allowing clinicians to make informed assessments of the patient's CI score 276 with ease.

Still further, metrics used for training a machine learning model to identify core metrics pertaining to gait data, may comprise entering: 15 spatiotemporal gait metrics (e.g., step time, stride time, swing time, stance time, single and double support time, etc.), six pressure metrics (i.e., the average and average peak area under the curve during stance phase for each pressure group), six IMU metrics (i.e., average peak, and range, root mean squared error, and meanSD for the accelerometer and gyroscope), and their respective variability metrics when appropriate (i.e., asymmetry, and standard deviation), resulting in 128 gait metrics.

When appropriate, each metric's average and standard deviation between feet and across the 10-second segment were calculated, resulting in 90 metrics.

A machine learning model may, according to some embodiments, identify 22 core metrics to separate MS patients from a healthy control population. This example shows identified 4 core metrics 2002, heel asymmetry 2004, single support percent 2006, swing time asymmetry 2008, toe asymmetry 2012.

In the illustrated embodiment, it can be seen that the MS patient reference population 2014 gait is highly consistent with MS population but significantly different from the healthy participants.

Further, by analyzing the box plot's whiskers 2026 for the MS population, it can be seen that the variability in PwMS is much greater than in healthy people, resulting in many challenges when categorizing data without a significant and robust database comprising a variety of test environments and movement activity to train the ml algorithms 144.

The present disclosure comprises novel systems and methods for measuring and monitoring gait. The present disclosure incorporates a plurality of data layers to calculate gait metrics in the wild (i.e., activity recognition, gait detection, assistive device detection). Furthermore, the present disclosure teaches systems and methods for detecting assistive device usage, phenotyping in the wild, and assessing prolonged walking and how that changes phenotypes/spatiotemporal metrics.

Further to the novel systems and methods for measuring and monitoring gait is a novel system and method for evaluating gait in the form of a practical, actionable, SVM-trained CI score for gait quality specific to neurological disorders, as well as longitudinal tracking of CI scores compared to baseline values. Insights and analyses may also be determined and displayed.

In still further embodiments of the disclosure, there is taught unique user interfaces with patient and clinician environments and portals, where CI scores, their meanings, and their methods of calculation may be displayed.

In still further embodiments of the disclosure, there is taught unique user interfaces for patients which may automatically trigger reminders for exercises, measure exercise data, and send the measured data to cloud computing devices for processing and transformation into a score and assessment.

In accordance with one or more embodiments of the disclosure, there are described systems and methods for the development of a data-driven scoring system for MS that provides more insights and more clarity to clinicians than existing scales (e.g., EDSS).

It will be appreciated that, while not explicitly considered in the examples described above, in some embodiments the CI score may be tailored or adapted to be specific to a patient's demographics. Parameters that may be considered include, without limitation: age, sex, location/geography, ethnicity or the like. In some embodiments, the CI score may be adapted to be disease specific as well.

Neurological conditions can be negatively impacted by weather (e.g., hot/cold temperatures, humidity, ultraviolet ray intensity, etc.). Further, terrain (e.g., elevation, ground type, slope etc.) has been found to alter an individual's gait pattern. Therefore, it is important to consider environmental conditions when evaluating gait quality in the wild. In some embodiments, the system of the present disclosure, in accordance with different embodiments, may use positional data such as GPS data obtained from the user's smartphone to retrieve relevant weather and terrain information.

In some embodiments, the computer device 112 may be communicatively coupled to one or more databases operable to provide, based at least in part on the user's location, weather and/or environmental information (i.e., terrain, weather, ambient temperature, humidity, etc.).

In certain embodiments, the weather and terrain information may be provided and displayed to the user and clinician on a user device to provide additional context when presenting the CI. In certain embodiments, the weather and terrain information may be displayed on the user device in the form of a dialogue box. In certain embodiments, the weather and terrain information may be used as a scaling factor applied to the CI to reduce the influence of weather and terrain on the user's gait quality.

In some embodiments, the system of the present disclosure may be configured to provide feedback to users or patients in the form of CI trend data only (increasing, decreasing, constant), without including the specific CI scores. This allows patients to see, for example, if their walking quality is improving, deteriorating, or staying the same, without bringing to the patient's attention the fact that their walking quality may be significantly worse than a healthy control.

Many of the functional units described in this specification have been labeled as “engines”, or “modules”, and/or “buttons” in order to more particularly emphasize their implementation independence. For example, an engine or module may be implemented as a hardware circuit comprising custom VLSI circuits or gate arrays, off-the-shelf semiconductors such as logic chips, transistors, or other discrete components. An engine may also be implemented in programmable hardware devices such as field programmable gate arrays, programmable array logic, programmable logic devices or the like.

Engines and modules may also be implemented in software for execution by various types of processors. An identified engine of executable code may, for instance, comprise one or more physical or logical blocks of computer instructions which may, for instance, be organized as an object, procedure, or function. Nevertheless, the executables of an identified engine need not be physically located together, but may comprise disparate instructions stored in different locations which, when joined logically together, comprise the engine or module and achieve the stated purpose for the module.

Indeed, an engine or module of executable code may be a single instruction, or many instructions, and may even be distributed over several different code segments, among different programs, and across several memory devices. Similarly, operational data may be identified and illustrated herein within engines, and may be embodied in any suitable form and organized within any suitable type of data structure. The operational data may be collected as a single data set, or may be distributed over different locations including over different storage devices, and may exist, at least partially, merely as electronic signals on a system or network. Where an engine or portions of an engine are implemented in software, the software portions are stored on one or more non-transitory, computer readable storage media.

The term “memory”, “machine-readable storage medium” or “computer readable medium” as used herein refers to any medium or media that participates in providing instructions to a processor for execution. Such a medium may take many forms, including, but not limited to, non-volatile media, volatile media, and transmission media. Non-volatile media include, for example, optical or magnetic disks, such as data storage devices. Volatile media include dynamic memory, such as random access memory (RAM). Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

The term “processor” includes general-purpose microprocessors, microcontrollers, a Digital Signal Processors (DSP), Application Specific Integrated Circuits (ASIC), Field Programmable Gate Arrays (FPGA), Programmable Logic Devices (PLD), discrete hardware components, or any other suitable entity that can perform calculations or other manipulations of information. One or more processors in a multi-processing arrangement may also be employed to execute the sequences of instructions contained in a memory. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., such as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Information as herein shown and described in detail is fully capable of attaining the above-described object of the present disclosure, the presently preferred embodiment of the present disclosure, and is, thus, representative of the subject matter which is broadly contemplated by the present disclosure.

What is claimed is:

1. A computer implemented method for evaluating a user's gait, the method comprising:
receiving, by a computing device having a memory and a processor, from a pair of smart insoles communicatively coupled to the computing device and worn by the user, data measured by one or more types of sensors;
segmenting, by the processor of the computing device, the data into gait-related segmented data comprising one or more of: gait cycle segmentation or activity type;
processing the gait-related segmented data, via one or more algorithms stored in the memory of the computing device, to determine one or more of:
gait patterns associated with the segmented data;
gait parameters associated with the segmented data;
gait phenotypes associated with the segmented data;
calculating, via one or more algorithms a composite gait quality score, based on a combination and interaction of at least two of: the segmented data, the gait patterns, the gait parameters, and the gait phenotypes.

2. The computer implemented method of claim 1, further comprising:
displaying, on a graphical user interface of a user device communicatively coupled to the computing device, the composite gait quality score.

3. The computer implemented method of claim 2, wherein the graphical user interface comprises one or more of: a clinician portal and a patient smart phone app;
wherein the clinician portal and patient app are further configured to receive raw and processed patient data from the system.

4. The computer implemented method of claim 1, wherein determining, via one or more algorithms a composite movement quality score, further comprises:
training a support vector machine (SVM), of the one or more algorithms, to classify walking data associated with a plurality of participants in one or more groups.

5. The computer implemented method of claim 4, wherein training the SVM comprises a feature selection step to remove redundant features and identify one or more designated metrics for evaluating gait in a specific patient population.

6. The computer implemented method of claim 1, wherein the one or more sensor types comprise at least one of: an accelerometer, a gyroscope, a magnetometer, a pressure sensor, and a temperature sensor.

7. The computer implemented method of claim 1, wherein the parameters comprise gait metrics from said heel strike, foot on floor, heel raise and toe off data.

8. The computer implemented method of claim 1, wherein the gait patterns comprise a gait signature of the individual from said gait metrics.

9. The computer implemented method of claim 1, wherein determining the composite gait quality score further comprises:
determining a progression over time of the combination and interaction of at least two of: the segmented data, the gait patterns, the gait parameters, and the gait phenotypes.

10. The computer implemented method of claim 8, wherein determining the composite gait quality score further comprises:
an assessment of the user's gait signature and recommendations for improvements.

11. The computer implemented method of claim 1, wherein determining the composite gait quality score further comprises:
an assessment of a change in a patient's neurological disease condition based on measured changes to the patient's gait.

12. The computer implemented method of claim 1, wherein determining the composite gait quality score further comprises:
determining a baseline objective walking quality score of the individual obtained prior to start of a rehabilitation program or immediately following an injury;
determining, based on the identified changes in gait pattern, an effectiveness of said rehabilitation program.

13. The computer-implemented method of claim 1, wherein said processing is optimized, by the processor, based on a type of assistive device being used by the user.

14. A system for evaluating a user's gait, the system comprising:
a processor; and
a memory comprising instructions stored thereon, which when executed by the processor, causes the processor to:
receive data measured by one or more types of sensors of a pair of smart insoles worn by the user, the smart insoles in communication with the computing device, segment the data into gait-related segmented data comprising one or more of: gait cycle segmentation or activity type;
process the gait-related segmented data, via one or more algorithms stored in the memory of the computing device, to determine one or more of:
gait patterns associated with the segmented data;
gait parameters associated with the segmented data;
gait phenotypes associated with the segmented data;
calculate, via one or more algorithms, a composite gait quality score, based on a combination and interaction of at least two of: the segmented data, the gait patterns, the gait parameters, and the gait phenotypes.

15. The system of claim 14, further comprising a user device communicatively coupled to the processor, and operable to display on a graphical user interface the composite gait quality score.

16. The system of claim 14 wherein determining, via one or more algorithms a composite gait quality score, further comprises:
training a support vector machine (SVM), of the one or more algorithms, to classify walking data associated with a plurality of participants in one or more groups.

17. The system of claim 16, wherein training the SVM comprises a feature selection step to remove redundant features and identify one or more designated metrics for evaluating gait.

18. The system of claim 14, wherein said determining is optimized based on a type of assistive device used by the user.

19. A non-transitory computer-readable storage medium comprising instructions stored thereon, which when executed by one or more processors, cause the one or more processors to perform operations for evaluating a user's gait, the operations comprising:
receiving data, measured by one or more sensors of a pair of smart insoles worn by the user, the smart insoles in communication with the computing device,
segmenting, by a processor of the computing device, the data into gait-related segmented data comprising one or more of: heel strike, foot on floor, heel raise, toe off data, and activity type;
processing the gait-related segmented data, via one or more algorithms stored in the memory of the computing device, to determine one or more of:
gait patterns associated with the segmented data;
gait parameters associated with the segmented data;
gait phenotypes associated with the segmented data;
calculating, via one or more algorithms a composite gait quality score, based on a combination and interaction of at least two of: the segmented data, the gait patterns, the gait parameters, and the gait phenotypes.

20. The non-transitory computer-readable storage medium of claim 19,
wherein determining, via one or more algorithms a composite gait quality score, further comprises:
training a support vector machine (SVM), of the one or more algorithms, to classify walking data associated with a plurality of participants in one or more groups.
